SEQUENCE LISTING (91 sequences)

SEQ ID NO: 1 - DNA, 82 bp, Artificial Sequence
  Synthetic construct
  misc_feature (26)..(33): n is a, c, g, or t
agtcactatccagaccaaagcgcccnnnnnnnncttcagcttcccgatatccgacggtagtgtcagatggaaaagatggtct

SEQ ID NO: 2 - DNA, 83 bp, Artificial Sequence
  Synthetic construct
  misc_feature (27)..(34): n is a, c, g, or t
ggatggtggtgcatggccgttcttagnnnnnnnncttcagcttcccgatatccgacggtagtgtagaaattgacggaagggca

SEQ ID NO: 3 - DNA, 81 bp, Artificial Sequence
  Synthetic construct
  misc_feature (24)..(31): n is a, c, g, or t
tgcgcctacaagcggtcggagctnnnnnnnncttcagcttcccgatatccgacggtagtgtttctgcgtgaatctgccggg

SEQ ID NO: 4 - DNA, 78 bp, Artificial Sequence
  Synthetic construct
  misc_feature (20)..(27): n is a, c, g, or t
gcttaacacatgcaagtcgnnnnnnnncttcagcttcccgatatccgacggtagtgttaataggtatttgaataaggt

SEQ ID NO: 5 - DNA, 78 bp, Artificial Sequence
  Synthetic construct
  misc_feature (21)..(28): n is a, c, g, or t
tgaccttattcaaatacctannnnnnnncttcagcttcccgatatccgacggtagtgttcgacttgcatgtgttaagc

SEQ ID NO: 6 - DNA, 83 bp, Artificial Sequence
  Synthetic construct
  misc_feature (25)..(32): n is a, c, g, or t
cgtaacaaggtagccgtaccggaannnnnnnncttcagcttcccgatatccgacggtagtgtaatgcgttcccgggccttgta

SEQ ID NO: 7 - DNA, 82 bp, Artificial Sequence
  Synthetic construct
  misc_feature (20)..(27): n is a, c, g, or t
gtagtcatatgcttgtctcnnnnnnnncttcagcttcccgatatccgacggtagtgttggctagttgtagagagtagtaaaa

SEQ ID NO: 8 - DNA, 83 bp, Artificial Sequence
  Synthetic construct
  misc_feature (28)..(35): n is a, c, g, or t
acggcccaccaaagcgacgatcagtagnnnnnnnncttcagcttcccgatatccgacggtagtgtctgggggataacagttag

SEQ ID NO: 9 - DNA, 79 bp, Artificial Sequence
  Synthetic construct
  misc_feature (22)..(29): n is a, c, g, or t
caaggtttccgtaggtgaaccnnnnnnnncttcagcttcccgatatccgacggtagtgtaccgcccgtcgctactaccg

SEQ ID NO: 10 - DNA, 81 bp, Artificial Sequence
  Synthetic construct
  misc_feature (21)..(28): n is a, c, g, or t
ggtggtgcatggctgtcgtcnnnnnnnncttcagcttcccgatatccgacggtagtgtaattgacggggccccgcacaagc

SEQ ID NO: 11 - DNA, 82 bp, Artificial Sequence
  Synthetic construct
  misc_feature (23)..(30): n is a, c, g, or t
cggcggccgtaactataacggtnnnnnnnncttcagcttcccgatatccgacggtagtgtcccaggcaactgtttatcaaaa

SEQ ID NO: 12 - DNA, 81 bp, Artificial Sequence
  Synthetic construct
  misc_feature (24)..(31): n is a, c, g, or t
gtgggcagtttgactggggcggtnnnnnnnncttcagcttcccgatatccgacggtagtgttgtacaggataggtgggaga

SEQ ID NO: 13 - DNA, 83 bp, Artificial Sequence
  Synthetic construct
  misc_feature (29)..(36): n is a, c, g, or t
gcaaggttgaaactcaaaggaattgacgnnnnnnnncttcagcttcccgatatccgacggtagtgttagcaaacaggattaga

SEQ ID NO: 14 - DNA, 82 bp, Artificial Sequence
  Synthetic construct
  misc_feature (29)..(36): n is a, c, g, or t
gataggttgggggtgtacgcgcagtaatnnnnnnnncttcagcttcccgatatccgacggtagtgtgttgtcacgccagtgg

SEQ ID NO: 15 - DNA, 83 bp, Artificial Sequence
  Synthetic construct
  misc_feature (26)..(33): n is a, c, g, or t
cactgttttgattttttacaccttgnnnnnnnncttcagcttcccgatatccgacggtagtgtttacgcgttactcacccgtc

SEQ ID NO: 16 - DNA, 83 bp, Artificial Sequence
  Synthetic construct
  misc_feature (30)..(37): n is a, c, g, or t
ggttttctgcgttcagcctgagaagggggnnnnnnnncttcagcttcccgatatccgacggtagtgtatggggcaaaagacgt

SEQ ID NO: 17 - DNA, 82 bp, Artificial Sequence
  Synthetic construct
  misc_feature (24)..(31): n is a, c, g, or t
gtggttatcctgcgttgatgcctnnnnnnnncttcagcttcccgatatccgacggtagtgtccacaagaacacatcatacaa

SEQ ID NO: 18 - DNA, 83 bp, Artificial Sequence
  Synthetic construct
  misc_feature (24)..(31): n is a, c, g, or t
gtaaggtgcagaaagaatatgcannnnnnnncttcagcttcccgatatccgacggtagtgtctctgtgttagtttaaagtgca

SEQ ID NO: 19 - DNA, 81 bp, Artificial Sequence
  Synthetic construct
  misc_feature (28)..(35): n is a, c, g, or t
ggggagtacggccgcaaggttgaaactnnnnnnnncttcagcttcccgatatccgacggtagtgtgcgaaagcgtgggtag

SEQ ID NO: 20 - DNA, 82 bp, Artificial Sequence
  Synthetic construct
  misc_feature (25)..(32): n is a, c, g, or t
aatgaaaacgtccttggcaaatgcnnnnnnnncttcagcttcccgatatccgacggtagtgttgatttctcgtaaggtgccg

SEQ ID NO: 21 - DNA, 83 bp, Artificial Sequence
  Synthetic construct
  misc_feature (21)..(28): n is a, c, g, or t
gcacgagctgacgacaaccannnnnnnncttcagcttcccgatatccgacggtagtgttgctgatttgacgtcatccccacct

SEQ ID NO: 22 - DNA, 78 bp, Artificial Sequence
  Synthetic construct
  misc_feature (25)..(32): n is a, c, g, or t
gggggattagctcagttggctagannnnnnnncttcagcttcccgatatccgacggtagtgtggaaggtgcggctgga

SEQ ID NO: 23 - DNA, 83 bp, Artificial Sequence
  Synthetic construct
  misc_feature (21)..(28): n is a, c, g, or t
ccctgtcacagaactgccgcnnnnnnnncttcagcttcccgatatccgacggtagtgttccaaaggaactcctaccttacgcc

SEQ ID NO: 24 - DNA, 81 bp, Artificial Sequence
  Synthetic construct
  misc_feature (27)..(34): n is a, c, g, or t
gactctaaatatttaatcaaatacctnnnnnnnncttcagcttcccgatatccgacggtagtgtccttcaacaggacatca

SEQ ID NO: 25 - DNA, 78 bp, Artificial Sequence
  Synthetic construct
  misc_feature (21)..(28): n is a, c, g, or t
tggaacaggacgtcatagagnnnnnnnncttcagcttcccgatatccgacggtagtgtaaaccaaccgggattgcctt

SEQ ID NO: 26 - DNA, 83 bp, Artificial Sequence
  Synthetic construct
  misc_feature (24)..(31): n is a, c, g, or t
gcatgatgatttgacgtcatcccnnnnnnnncttcagcttcccgatatccgacggtagtgtcttcatgtagtcgagttgcaga

SEQ ID NO: 27 - DNA, 83 bp, Artificial Sequence
  Synthetic construct
  misc_feature (27)..(34): n is a, c, g, or t
ggagagcgcctgctttgcacgcaggannnnnnnncttcagcttcccgatatccgacggtagtgtaagtcgtaacaaggtagcc

SEQ ID NO: 28 - DNA, 83 bp, Artificial Sequence
  Synthetic construct
  misc_feature (30)..(37): n is a, c, g, or t
cagcgttcgtcctgagccaggatcaaactnnnnnnnncttcagcttcccgatatccgacggtagtgttccaatagttatcccc

SEQ ID NO: 29 - DNA, 83 bp, Artificial Sequence
  Synthetic construct
  misc_feature (30)..(37): n is a, c, g, or t
gatacatagccgacctgagagggtgatcgnnnnnnnncttcagcttcccgatatccgacggtagtgtagctaataccgcataa

SEQ ID NO: 30 - DNA, 81 bp, Artificial Sequence
  Synthetic construct
  misc_feature (26)..(33): n is a, c, g, or t
tttgatcctggctcaggacgaacgcnnnnnnnncttcagcttcccgatatccgacggtagtgtttgaaaactgaacaagac

SEQ ID NO: 31 - DNA, 82 bp, Artificial Sequence
  Synthetic construct
  misc_feature (26)..(33): n is a, c, g, or t
gccacggctaactacgtgccagcagnnnnnnnncttcagcttcccgatatccgacggtagtgtggacgaaagtctgacggag

SEQ ID NO: 32 - DNA, 19 bp, Artificial Sequence
  Synthetic construct
cagatggaaaagatggtct

SEQ ID NO: 33 - DNA, 25 bp, Artificial Sequence
  Synthetic construct
agtcactatccagaccaaagcgccc

SEQ ID NO: 34 - DNA, 19 bp, Artificial Sequence
  Synthetic construct
agaaattgacggaagggca

SEQ ID NO: 35 - DNA, 26 bp, Artificial Sequence
  Synthetic construct
ggatggtggtgcatggccgttcttag

SEQ ID NO: 36 - DNA, 20 bp, Artificial Sequence
  Synthetic construct
ttctgcgtgaatctgccggg

SEQ ID NO: 37 - DNA, 23 bp, Artificial Sequence
  Synthetic construct
tgcgcctacaagcggtcggagct

SEQ ID NO: 38 - DNA, 21 bp, Artificial Sequence
  Synthetic construct
aatgcgttcccgggccttgta

SEQ ID NO: 39 - DNA, 24 bp, Artificial Sequence
  Synthetic construct
cgtaacaaggtagccgtaccggaa

SEQ ID NO: 40 - DNA, 25 bp, Artificial Sequence
  Synthetic construct
tggctagttgtagagagtagtaaaa

SEQ ID NO: 41 - DNA, 19 bp, Artificial Sequence
  Synthetic construct
gtagtcatatgcttgtctc

SEQ ID NO: 42 - DNA, 18 bp, Artificial Sequence
  Synthetic construct
ctgggggataacagttag

SEQ ID NO: 43 - DNA, 27 bp, Artificial Sequence
  Synthetic construct
acggcccaccaaagcgacgatcagtag

SEQ ID NO: 44 - DNA, 20 bp, Artificial Sequence
  Synthetic construct
accgcccgtcgctactaccg

SEQ ID NO: 45 - DNA, 21 bp, Artificial Sequence
  Synthetic construct
caaggtttccgtaggtgaacc

SEQ ID NO: 46 - DNA, 23 bp, Artificial Sequence
  Synthetic construct
aattgacggggccccgcacaagc

SEQ ID NO: 47 - DNA, 20 bp, Artificial Sequence
  Synthetic construct
ggtggtgcatggctgtcgtc

SEQ ID NO: 48 - DNA, 22 bp, Artificial Sequence
  Synthetic construct
cccaggcaactgtttatcaaaa

SEQ ID NO: 49 - DNA, 22 bp, Artificial Sequence
  Synthetic construct
cggcggccgtaactataacggt

SEQ ID NO: 50 - DNA, 20 bp, Artificial Sequence
  Synthetic construct
tgtacaggataggtgggaga

SEQ ID NO: 51 - DNA, 23 bp, Artificial Sequence
  Synthetic construct
gtgggcagtttgactggggcggt

SEQ ID NO: 52 - DNA, 22 bp, Artificial Sequence
  Synthetic construct
tagtgtgttgtcacgccagtgg

SEQ ID NO: 53 - DNA, 28 bp, Artificial Sequence
  Synthetic construct
gcaaggttgaaactcaaaggaattgacg

SEQ ID NO: 54 - DNA, 16 bp, Artificial Sequence
  Synthetic construct
gttgtcacgccagtgg

SEQ ID NO: 55 - DNA, 28 bp, Artificial Sequence
  Synthetic construct
gataggttgggggtgtacgcgcagtaat

SEQ ID NO: 56 - DNA, 20 bp, Artificial Sequence
  Synthetic construct
ttacgcgttactcacccgtc

SEQ ID NO: 57 - DNA, 24 bp, Artificial Sequence
  Synthetic construct
cactgttttgattttttacacctt

SEQ ID NO: 58 - DNA, 16 bp, Artificial Sequence
  Synthetic construct
atggggcaaaagacgt

SEQ ID NO: 59 - DNA, 29 bp, Artificial Sequence
  Synthetic construct
ggttttctgcgttcagcctgagaaggggg

SEQ ID NO: 60 - DNA, 21 bp, Artificial Sequence
  Synthetic construct
ccacaagaacacatcatacaa

SEQ ID NO: 61 - DNA, 23 bp, Artificial Sequence
  Synthetic construct
gtggttatcctgcgttgatgcct

SEQ ID NO: 62 - DNA, 22 bp, Artificial Sequence
  Synthetic construct
ctctgtgttagtttaaagtgca

SEQ ID NO: 63 - DNA, 23 bp, Artificial Sequence
  Synthetic construct
gtaaggtgcagaaagaatatgca

SEQ ID NO: 64 - DNA, 16 bp, Artificial Sequence
  Synthetic construct
gcgaaagcgtgggtag

SEQ ID NO: 65 - DNA, 27 bp, Artificial Sequence
  Synthetic construct
ggggagtacggccgcaaggttgaaact

SEQ ID NO: 66 - DNA, 20 bp, Artificial Sequence
  Synthetic construct
tgatttctcgtaaggtgccg

SEQ ID NO: 67 - DNA, 24 bp, Artificial Sequence
  Synthetic construct
aatgaaaacgtccttggcaaatgc

SEQ ID NO: 68 - DNA, 25 bp, Artificial Sequence
  Synthetic construct
tgctgatttgacgtcatccccacct

SEQ ID NO: 69 - DNA, 20 bp, Artificial Sequence
  Synthetic construct
gcacgagctgacgacaacca

SEQ ID NO: 70 - DNA, 16 bp, Artificial Sequence
  Synthetic construct
ggaaggtgcggctgga

SEQ ID NO: 71 - DNA, 24 bp, Artificial Sequence
  Synthetic construct
gggggattagctcagttggctaga

SEQ ID NO: 72 - DNA, 25 bp, Artificial Sequence
  Synthetic construct
tccaaaggaactcctaccttacgcc

SEQ ID NO: 73 - DNA, 20 bp, Artificial Sequence
  Synthetic construct
ccctgtcacagaactgccgc

SEQ ID NO: 74 - DNA, 17 bp, Artificial Sequence
  Synthetic construct
ccttcaacaggacatca

SEQ ID NO: 75 - DNA, 26 bp, Artificial Sequence
  Synthetic construct
gactctaaatatttaatcaaatacct

SEQ ID NO: 76 - DNA, 20 bp, Artificial Sequence
  Synthetic construct
aaaccaaccgggattgcctt

SEQ ID NO: 77 - DNA, 20 bp, Artificial Sequence
  Synthetic construct
tggaacaggacgtcatagag

SEQ ID NO: 78 - DNA, 22 bp, Artificial Sequence
  Synthetic construct
cttcatgtagtcgagttgcaga

SEQ ID NO: 79 - DNA, 23 bp, Artificial Sequence
  Synthetic construct
gcatgatgatttgacgtcatccc

SEQ ID NO: 80 - DNA, 19 bp, Artificial Sequence
  Synthetic construct
aagtcgtaacaaggtagcc

SEQ ID NO: 81 - DNA, 26 bp, Artificial Sequence
  Synthetic construct
ggagagcgcctgctttgcacgcagga

SEQ ID NO: 82 - DNA, 16 bp, Artificial Sequence
  Synthetic construct
tccaatagttatcccc

SEQ ID NO: 83 - DNA, 29 bp, Artificial Sequence
  Synthetic construct
cagcgttcgtcctgagccaggatcaaact

SEQ ID NO: 84 - DNA, 16 bp, Artificial Sequence
  Synthetic construct
agctaataccgcataa

SEQ ID NO: 85 - DNA, 29 bp, Artificial Sequence
  Synthetic construct
gatacatagccgacctgagagggtgatcg

SEQ ID NO: 86 - DNA, 18 bp, Artificial Sequence
  Synthetic construct
ttgaaaactgaacaagac

SEQ ID NO: 87 - DNA, 25 bp, Artificial Sequence
  Synthetic construct
tttgatcctggctcaggacgaacgc

SEQ ID NO: 88 - DNA, 19 bp, Artificial Sequence
  Synthetic construct
ggacgaaagtctgacggag

SEQ ID NO: 89 - DNA, 25 bp, Artificial Sequence
  Synthetic construct
gccacggctaactacgtgccagcag

SEQ ID NO: 90 - DNA, 20 bp, Artificial Sequence
  Synthetic construct
tcgacttgcatgtgttaagc

SEQ ID NO: 91 - DNA, 20 bp, Artificial Sequence
  Synthetic construct
tgaccttattcaaataccta

(19) United States
(12) Patent Application Publication
(10) Pub. No.: US 2024/0229164 A1
(43) Pub. Date: Jul. 11, 2024

(54) RNA Profiling of the microbiome

(71) Applicant: STICHTING RADBOUD UNIVERSITAIR MEDISCH CENTRUM, Nijmegen (NL)

(72) Inventors: Wilhelmus Petrus Johannes Leenders, Nijmegen (NL); Wilhelmus Johannes Gerardus Melchers, Nijmegen (NL); Martijn Adriaan Huijnen, Nijmegen (NL)

(73) Assignee: STICHTING RADBOUD UNIVERSITAIR MEDISCH CENTRUM, Nijmegen (NL)

(21) Appl. No.: 18/563,884

(22) PCT Filed: May 31, 2022
(86) PCT No.: PCT/EP2022/064741

§ 371 Date: Nov. 23, 2023

(30) Foreign Application Priority Data
May 31, 2021 (EP) 21176843.7

(51) Int. Cl. C12Q 1/689; C12Q 1/6874

(52) U.S. Cl. CPC C12Q 1/689; C12Q 1/6874; C12Q 2600/118; C12Q 2600/16

(57) ABSTRACT

The present invention relates to the field of medicine and molecular diagnostics. In particular, it relates to a molecular profiling assay for a complex microbiome.

### FIELD OF THE INVENTION

The present invention relates to the field of medicine and molecular diagnostics. In particular, it relates to a molecular profiling assay for a complex microbiome.

### BACKGROUND OF THE INVENTION

High-risk human papillomavirus (hrHPV)-induced cervical cancer affects more than half a million women every year [1]. Although the oncogenic role of hrHPV is clear in this process, only a minority of hrHPV infections lead to cervical lesions, and ultimately, cancer. Hence, there is a need to better understand hrHPV-induced alterations in the cervicovaginal environment that contribute to cancer development. Accordingly, recent efforts have focused on the host immune response and the cervicovaginal microbiome (CVM) [2-4]. The latter has a significant role in women's cervical health and disease [5]. Throughout women's lives, the CVM can change during the menstrual cycle, pregnancy, or after sexual activities [6-10]. Such microbiome changes also occur in pathogenic conditions like bacterial vaginosis (BV), Candidiasis, and viral infections [11-13]. Interestingly, the composition of the CVM is characterized by microbial dominancy and diversity, creating characteristic community state types (CST) that could be either dominant for Lactobacillus species (CST I, II, III, and V) or diverse for other bacterial species (CST IV). Variations in the CVM have been widely described in relation to hrHPV infections, with CST IV being significantly associated with high-grade cervical lesions and cancer [4, 14, 15]. Furthermore, recent investigations have determined that these microbiome alterations not only occur at the genus level but also at the species level, suggesting that specific microbial species and CST are associated with progressive or regressive behavior of cervical lesions and could act as biomarkers for the disease [16-18]. Nevertheless, studying the CVM and elucidating its function currently relies on detection methods such as short length 16S rRNA gene sequencing, which are unable to distinguish microbes at this taxonomic rank [19-22].

Currently, microbiome profiling is mostly performed by 16S rRNA gene sequencing (16S rRNA-seq). This technology is based on the sequence analysis of hypervariable regions (VRs) in ribosomal 16S rRNA genes for microbes identification [23, 24]. PCR amplicons covering two VRs (e.g., V1-V2, V3-V4, etc.) are generated with degenerate primer sets and subjected to next-generation sequencing. The technique results in bacterial identification mostly at the genus level [25]. However, several studies have observed bias in microbiome profiling with 16S rRNA-seq due to variability in the selection of primers and VRs for amplification and sequencing [26-28]. Since changes in the CVM also take place at the species level, it is essential to develop detection methods with higher resolution and specificity.

### DESCRIPTION OF THE FIGURES

FIG. 1. Schematic representation of the MIP selection as described herein.

FIG. 2. CiRNAseq exhibits high specificity and resolution. A) CiRNAseq exhibits high specificity in a mixed microbial sample. The method can discriminate different microbes in a single sample of mixed bacteria. B) CiRNAseq displays high-resolution in detecting microbes. The technique can identify different species of the same genus such as P. copri, P. denticola, and P. disiens and other species from a distinctive genus such as L. delbruecki, L. fermentum, and L. jensenii within the same sample. The CVM panel was shortened in FIG. 2B (CVMPs) to only display species and isolates from Lactobacillus and Prevotella genera. Values represent the percentage of reactive smMIPs in the specific set for each microbe. Negative control: water.

FIG. 3. CiRNAseq RNA quantification capacity mirrors bacterial growth and activity. A) The OD obtained from monitoring E. coli growth for 48 hours reveals the bacterial growth phases. The nine orange time points indicate the phases from when samples were taken for sequencing analyses. B) E. coli URC correlated with the OD, particularly from the lag to the exponential phases. Samples taken in the stationary phase had lower URC than the last measurement within the exponential phase. C) RNA concentrations of the samples taken for sequencing are parallel to the OD, indicating that low URC found in time points six and seven may reflect the measurement of ribosomal activity. D) RNA concentrations also match the URC obtained from sequencing.

FIG. 4. CiRNAseq holds a deeper sequencing performance than 16S rRNA-seq. A) 16S rRNA-seq (SN-A) and CiRNAseq (SN-B) possess similar sequencing capacity when differentiating 34 of the 43 genera analyzed. The methods gave the same results with respect to quantifying the genera Lactobacillus and Gardnerella when analyzing cervical smears. The microbial composition of samples A and B is similar when analyzed using the two sequencing techniques. Microbial species and isolates URC were summed to show the results at the genus level. B) CiRNAseq allows detecting bacteria at high-resolution. The technique suggested 24 different bacteria species, including two species of Anaerococcus, seven Lactobacillus species, five species of Prevotella, and two species of Sneathia. For FIG. 4A, values represent the relative abundances of each microbe in the sample. Bacteria species isolates within our CVMP were considered for display of FIG. 4B.

FIG. 5. CiRNAseq: the CVM shifts upon hrHPV infection. A) Unsupervised clustering analysis of randomly selected hrHPV negative and hrHPV positive women (CIN2+) shows three distinct clusters from left to right: the first cluster (1) includes a higher proportion of hrHPV negative women, who have a microbiome characterized of Lactobacillus species, and particularly L. crispatus (CST I). The second cluster (2) contains a higher proportion of hrHPV positive women with CIN2+ lesions, who possess a diverse microbiome (CST IV) containing distinctive bacteria such as Atopobium vaginae, Dialister micraerophilus, Gardnerella vaginalis, Lactobacillus iners, Megasphaera genomosp type 1, Sneathia amnii, and Sneathia sanguinegens. The third cluster (3) includes both hrHPV negative and hrHPV positive women with predominantly hrHPV negative women, who have a unique microbiome characterized by Lactobacillus species such as L. gasseri (CST II), L. iners (CST III), L. jensenii (CST V), and L. acidophilus. Clustering distance for columns: Canberra; clustering method: Ward (unsquared distances); Row scaling: Pareto scaling. The CVMP was shortened (CVMPs) to only include species with URC. URC from bacteria isolates in our CVMP were considered for analysis. B) Principal Component Analysis (PCA) shows that hrHPV negative and hrHPV positive samples are correlated with PC1. The loading score of PC1 (data not shown) indicates that anaerobic bacteria have the stronger association with PC1 (Additional File 4). Original values are In(x+1)-transformed. No scaling is applied to rows; SVD with imputation is used to calculate principal components. C) Histogram of the LDA scores computed for features differentially abundant between hrHPV negative (negative) and hrHPV positive women (positive).

FIG. 6. CiRNAseq profiling reveals alterations in the CVM.

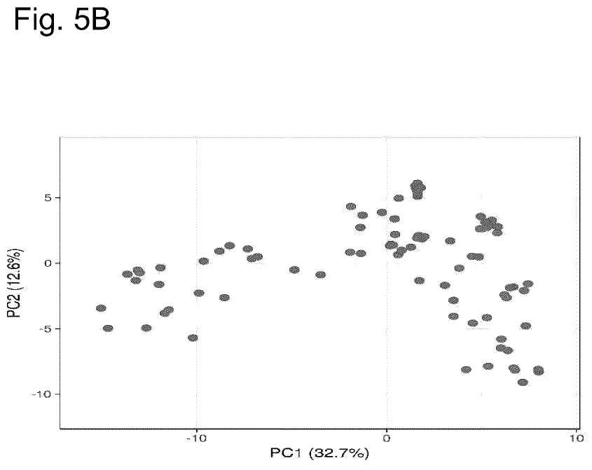

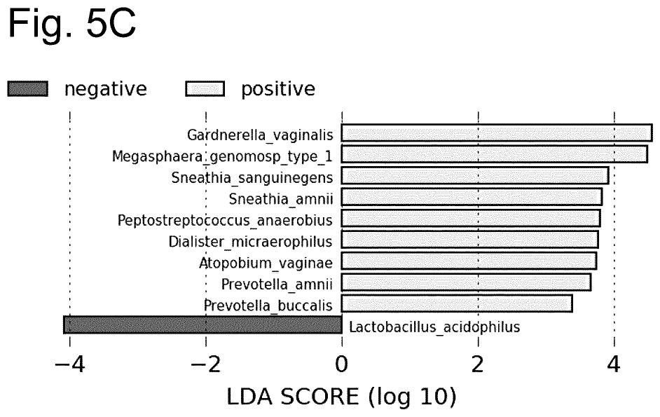

A) The alteration of the microbial diversity at the species level reflects the need for high-resolution sequencing methods. Cluster 1 enriched for CST I and hrHPV negative women has a less diverse CVM with characteristic Lactobacillus species. In contrast, cluster 2 enriched for CST IV and hrHPV positive women with CIN2+ contain various microbial species in their microbiome. CST I and IV are derived from the analysis detailed in FIG. 5A. Bacteria isolates from our CVMP were considered for only three species: G. vaginalis, L. johnsonii, and Ureaplasma parvum. Species richness (B) and Shannon's diversity index (C) further confirms the increase in microbial diversity in hrHPV positive women. It also demonstrates that hrHPV infections correlate with a rich and diverse CVM. D) Using CiRNAseq, we can quantify microbial species within the CVM. L. iners is less abundant in hrHPV positive women than in hrHPV negative women, indicating that the progression of hrHPV infections to high-grade cervical lesions is associated with a decreased relative abundance of L. iners. Samples were selected from our cohort of 92 samples. Negative: negative for hrHPV; Positive: positive for hrHPV; *, p<0.05.

### DETAILED DESCRIPTION OF THE INVENTION

Circular probe-based RNA sequencing (CiRNAseq) using single-molecule molecular inversion probes (smMIPs) has proven to be a useful tool for cancer research [30-33] and hrHPV expression studies [34]. smMIPs can be designed to target any nucleic acid sequence and thus could be applied to recognize multiple VRs and to identify diverse microbes such as bacteria, fungi, and viruses simultaneously. Likewise, by targeting and combining multiple VRs for microbiome profiling, CiRNAseq could perform high-resolution sequencing with high specificity and sensitivity [29]. Besides being customizable for its targets, the addition of a unique molecule identifier (UMI) to a smMIP makes the counting of amplified smMIPs possible, which could also be valuable for absolute microbiome RNA or DNA quantification [30, 34]. Because CiRNAseq uses barcode technology, it can handle hundreds of samples in one sequencing run, making the technique cost-effective. Furthermore, it requires fewer specialized skills for data analyses and interpretation than other sequencing methods such as 16S rRNA-seq, making it a handy and accessible technology [35, 36].

The inventors have developed a new method of using the CiRNAseq technique to enable high-resolution microbiome profiling. Using this technique, the inventors were able to develop a set of 30 smMIPs capable of targeting the 434 previously identified microbes that have been recognized as significant in the cervicovaginal environment enabling profiling of the entire cervicovaginal environment.

A preferred method of generating RNA profiles is by using smMIPs that can be designed with the published MIPGEN protocol (18) that selects optimal ligation and extension probe sequences that are predicted to hybridize against a cDNA of interest while leaving a gap between the ligation and extension parts of the probe. The ligation and extension parts of the probes may hybridize to any part of the cDNA, including sequences that are protein encoding and untranslated regions. The smMIPs are preferably designed to have extension and ligation probes that are fully homologous to 16S gene sequences of more than 1 species, but flank regions of interest that are heterologous in the different recognized species, thus allowing annotation of different species with only one smMIP. Extension and ligation parts of the probes are located on the same strand of cDNA, contrasting the situation with regular PCR, which uses probes that are directed at two different complementary strands.

A method, suitable for viruses and eukaryotic organisms. is to locate the ligation and extension parts of the probes in different exons of a cDNA, which allows detection of specific splice variants.

A preferred method according to the invention is to contact a library of designed smMIPs according to the invention, that may consist of any number of smMIPs, with a population of cDNA molecules. After an initial heating and denaturation step followed by cooling, each smMIP will hybridize to its target cDNA sequence. By incubating the mixture with a DNA polymerase enzyme, all four deoxynucleotides and DNA ligase in an appropriate buffer, the extension probe part of the MIP will be extended until the 5′ end of the ligation probe is reached. The DNA ligase will then covalently link the 3′ end of the extended extension probe part to the ligation probe part, producing a circular smMIP molecule.

In the next step, a method known to the person skilled in the art, is used to remove unreacted, linear smMIPs and cDNA from the reaction mixture by exonuclease treatment, leaving a purified library of circular smMIPs.

Using a forward and a reverse oligonucleotide primer that specifically anneal to the backbone sequence that connects the ligation and extension probes parts of the MIP, a PCR amplification of the gap sequence is performed. Preferably, one or both of the oligonucleotide primers that are used in this PCR is equipped with a barcode, allowing easy selection of all PCR products that are obtained from a specific sample. In a next step, the library of PCR amplicons are preferably analyzed on a next generation sequencing platform that yields FASTQ files containing information on nucleotide sequences of all PCR amplicons in the sample. Using an algorithm all PCR amplicons with the same barcode are grouped, producing a list of sequences for each individual cDNA sample.

Next, using another algorithm that uses the UMI, all identical PCR products will be considered to be derived from one originating smMIP. In this manner for each original RNA sample a list can be created that contains values that represent the original number of circularized smMIPs in the original library. This number is proportional to the number of cDNAs in the original sample. In a preferred method of interpretation, the values obtained for each individual smMIP are divided by the summated values of all smMIPs for each sample, followed by multiplying with a factor of one million, thus yielding a fragments per million value for each smMIP.

In a preferred method of interpretation, the mean FPM values of all different smMIPs that correspond to one transcript, are considered to be proportional to the number of transcripts that were present in the initial RNA sample of the analysis.

In another preferred method of interpretation, mean FPM values of individual transcripts are divided by mean FPM values of so-called house-keeping genes, to yield a relative abundance value of a transcript of interest.

In another preferred method, mean FPM values for transcripts from genes that are involved in metabolic pathways are used to deduce predominant metabolic pathways in a tissue.

A preferred method to analyze the FASTQ files further is to detect mutations in the next generation sequencing data. Preferably, mutations are considered as relevant if they are detected in more than two reads. The sequence information as provided in the FASTQ files should not be so narrowly construed as to require inclusion of erroneously identified bases. The skilled person is capable of identifying such erroneously identified bases and knows how to correct for such errors. A list of relevant mutations in a sample can be included in a database, preferably a standard query language (SQL)-based database that allows statistical analyses, for example by multivariate analysis.

Accordingly in a first aspect the invention provides for a method for in vitro profiling of a complex microbiome comprising:
providing a sample from the complex microbiome
performing RNA profiling on the sample by multiplex RNA sequencing, targeting multiple regions of interest. In certain embodiments, the region of interest is a gene of interest, or a part thereof.

Said method is herein referred to as the method according to the invention. “RNA profiling” is herein also referred to as targeted RNA sequencing of transcripts.

Preferably, in a method according to the invention, RNA profiling is performed by multiplex RNA sequencing, targeting multiple regions of interest. The sample RNA of interest may first be converted to copy-DNA (cDNA) using a method known in the art, such as using oligo-dT primers in case RNAs are polyadenylated, a mixture of random hexamer oligonucleotide primers, or a combination thereof.

In some embodiments, the sequenced RNA is mRNA, tRNA, rRNA or antisense RNA. Preferably, the method relates to multiplex mRNA sequencing and rRNA sequencing.

Preferably, in a method according to the invention, the multiplex RNA sequencing is performed using molecular inversion probes (MIPs), preferably MIPs comprising a detectable moiety, preferably a unique identifier sequence of a string of 3 to 10 random nucleotides (depicted as “N” in a sequence listing), more preferably a string of 3, more preferably 4, more preferably 5, more preferably 6, more preferably 7, most preferably 8, or preferably more than 8 random nucleotides (N) adjacent to the ligation part of the MIP or to the extension part of the MIP sequence (smMIPs).

The RNA of interest may be from human genes but may also be from genes of pathogens such as DNA viruses and RNA viruses, including but not limited to human immune deficiency virus (HIV); human papilloma viruses, including but not limited to the subtypes HPV6, HPV11, HPV16, HPV18, HPV31, HPV33, HPV35, HPV39, HPV45, HPV51, HPV52, HPV56, HPV58, HPV59, HPV66, HPV68, HPV73, HPV82; monkey pox virus, hepatitis A virus; hepatitis B virus; hepatitis C virus; hepatitis E virus; Ebola virus; Epstein Bar Virus (EBV); influenza viruses; West-Nile virus, chikungunya virus, polyoma virus; cytomegalovirus; rhinovirus, corona viruses, including but not limited to SARS-CoV, SARS-CoV2, MERS, HCoV-OC43, HCoC-NL63 but also genes from the category of oncolytic viruses that are known to persons skilled in the art to treat cancers.

The RNA of interest may be from microbes and pathogens. In certain embodiments the RNA of interest is from commensal microbes such as commensal bacteria. In certain embodiments, the RNA of interest is from pathogenic fungi. In certain embodiments, RNA of interest may also be from (pathogenic) bacteria, such as Lactobacillus, Gardnerella and Bifidobacterium. In certain embodiments the RNA of interest is from any of the microbes or pathogens selected from table I.

In one embodiment, the method according to the invention is for profiling bacterial DNA present in the complex microbiome. Preferably, the bacterial DNA is DNA from the bacteria listed in table II.

The region of interest targeted by the multiplex RNA may be a region that defines the bacterial, fungal or viral identity or a region that defines functional aspects of bacteria, fungi or viruses.

TABLE I
Identified species/strains

| | |
|---|---|
| Weissella koreensis | Lactobacillus fermentum ATCC 14931 |
| Abiotrophia defectiva ATCC 49176 | Lactobacillus fermentum CECT 5716 |
| Acidaminococcus intestini | Lactobacillus gallinarum |
| Actinobaculum massiliense | Lactobacillus gasseri |
| Actinomyces neuii | Lactobacillus gasseri 202-4 |
| Actinomyces odontolyticus ATCC 17982 | Lactobacillus gasseri 224-1 |
| Actinomyces sp. oral taxon 171 | Lactobacillus gasseri JV-V03 |
| Actinomyces urogenitalis DSM 15434 | Lactobacillus gasseri MV-22 |
| Aerococcus christensenii | Lactobacillus hamsteri |
| Akkermansia muciniphila ATCC BAA-835 | Lactobacillus helveticus |
| Alistipes putredinis DSM 17216 | Lactobacillus helveticus DSM 20075 = CGMCC |
| | 11877 |
| Alloprevotella rava | Lactobacillus iners |
| Alloprevotella tannerae ATCC 51259 | Lactobacillus iners AB-1 |
| Anaerococcus hydrogenalis | Lactobacillus iners DSM 13335 |
| Anaerococcus lactolyticus | Lactobacillus ingluviei |
| Anaerococcus obesiensis | Lactobacillus jensenii |
| Anaerococcus prevotii | Lactobacillus jensenii 269-3 |
| Anaerococcus tetradius | Lactobacillus jensenii 27-2-CHN |
| Anaerococcus vaginalis | Lactobacillus jensenii JV-V16 |
| Anaerotruncus colihominis DSM 17241 | Lactobacillus jensenii SJ-7A-US |
| Arcanobacterium haemolyticum DSM 20595 | Lactobacillus johnsonii |
| Aspergillus fumigatus | Lactobacillus johnsonii ATCC 33200 |
| Aspergillus oryzae | Lactobacillus johnsonii FI9785 |
| Aspergillus tubingensis | Lactobacillus kefiranofaciens |
| Atopobium parvulum DSM 20469 | Lactobacillus kitasatonis |
| Atopobium rimae ATCC 49626 | Lactobacillus mucosae |
| Atopobium vaginae | Lactobacillus oris |
| Bacteroides caccae ATCC 43185 | Lactobacillus plantarum |
| Bacteroides cellulosilyticus DSM 14838 | Lactobacillus pontis |
| Bacteroides coprocola DSM 17136 | Lactobacillus psittaci |
| Bacteroides coprophilus DSM 18228 = JCM | Lactobacillus reuteri |
| 13818 | |
| Bacteroides dorei 5_1_36/D4 | Lactobacillus reuteri 100 23 |
| Bacteroides dorei DSM 17855 | Lactobacillus reuteri CF48 3A |
| Bacteroides eggerthii DSM 20697 | Lactobacillus reuteri SD2112 |
| Bacteroides finegoldii DSM 17565 | Lactobacillus ruminis |
| Bacteroides fragilis | Lactobacillus salivarius CECT 5713 |
| Bacteroides fragilis 3_1_12 | Lactobacillus taiwanensis |
| Bacteroides fragilis 638R | Lactobacillus ultunensis DSM 16047 |
| Bacteroides intestinalis DSM 17393 | Lactobacillus vaginalis |
| Bacteroides ovatus ATCC 8483 | Lawsonella clevelandensis |
| Bacteroides ovatus SD CMC 3f | Leptotrichia buccalis C 1013 b |
| Bacteroides pectinophilus ATCC 43243 | Leptotrichia goodfellowii F0264 |
| Bacteroides plebeius DSM 17135 | Leptotrichia hofstadii F0254 |
| Bacteroides sp. 1_1_14 | Mageeibacillus indolicus |
| Bacteroides sp. 2_1_33B | Megasphaera genomosp type 1 |
| Bacteroides sp. 2_2_4 | Megasphaera micronuciformis |
| Bacteroides sp. 3_1_19 | Millerozyma farinosa |
| Bacteroides sp. 3_1_23 | Mobiluncus curtisii |
| Bacteroides sp. 3_1_33FAA | Mobiluncus curtisii ATCC 43063 |
| Bacteroides sp. 3_2_5 | Mobiluncus mulieris |
| Bacteroides sp. 4_3_47FAA | Mobiluncus mulieris 28-1 |
| Bacteroides sp. 9_1_42FAA | Mobiluncus mulieris ATCC 35243 |
| Bacteroides sp. D2 | Molluscum contagiosum virus |
| Bacteroides stercoris ATCC 43183 | Morganella morganii |
| Bacteroides thetaiotaomicron | Mycobacteroides abscessus |
| Bacteroides uniformis ATCC 8492 | Mycoplasma genitalium |
| Bacteroides vulgatus | Mycoplasma hominis |
| Bacteroides vulgatus PC510 | Nakaseomyces delphensis |
| Bifidobacterium adolescentis L2-32 | Naumovozyma castellii |
| Bifidobacterium angulatum JCM 7096 | Neisseria flavescens NRL30031 H210 |
| Bifidobacterium animalis subsp lactis BI-04 | Neisseria flavescens SK114 |
| Bifidobacterium animalis subsp lactis V9 | Neisseria gonorrhoeae |
| Bifidobacterium bifidum NCIMB 41171 | Neisseria meningitidis |
| Bifidobacterium breve | Neisseria mucosa ATCC 25996 |
| Bifidobacterium catenulatum | Neisseria sicca ATCC 29256 |
| Bifidobacterium dentium ATCC 27678 | Neisseria subflava NJ9703 |
| Bifidobacterium dentium ATCC 27679 | Olsenella uli DSM 7084 |
| Bifidobacterium dentium Bd1 | Oribacterium sinus F0268 |
| Bifidobacterium gallicum DSM20093 | Oribacterium sp. oral taxon 078 str. F0262 |
| Bifidobacterium kashiwanohense | Pantoea dispersa |
| Bifidobacterium longum | Parabacteroides distasonis ATCC 8503 |
| Bifidobacterium longum subsp infantis ATCC | Parabacteroides johnsonii DSM 18315 |
| 15697 = JCM 1222 = DSM 20088 | |
| Bifidobacterium longum subsp infantis CCUG | Parabacteroides merdae ATCC 43184 |
| 52486 | |
| Bifidobacterium longum subsp longum JDM301 | Parabacteroides sp. 2_1_7 |
| Bifidobacterium pseudocatenulatum | Parabacteroides sp. 20_3 |
| Bifidobacterium pseudocatenulatum DSM 20438 = | Parabacteroides sp. D13 |
| JCM 1200 = LMG 10505 | |
| Blautia hansenii DSM 20583 | Parastagonospora nodorum |
| Blautia obeum ATCC 29174 | Parvimonas micra |
| Bulleidia extructa W1219 | Peptoniphilus asaccharolyticus |
| Burkholderia cenocepacia J2315 | Peptoniphilus coxii |
| Burkholderiales bacterium 1_1_47 | Peptoniphilus duerdenii |
| Butyrivibrio crossotus DSM 2876 | Peptoniphilus grossensis |
| Campylobacter concisus 13826 | Peptoniphilus harei |
| Campylobacter hominis ATCC BAA-381 | Peptoniphilus lacrimalis |
| Campylobacter ureolyticus | Peptoniphilus sp. oral taxon 386 str. F0131 |
| Candida albicans | Peptoniphilus sp. oral taxon 836 str. F0141 |
| Candida castellii | Peptostreptococcus anaerobius |
| Candida dubliniensis | Phascolarctobacterium succinatutens |
| Candida glabrata | Phytophthora infestans |
| Candida maltosa | Porphyromonas endodontalis ATCC 35406 |
| Candida orthopsilosis | Porphyromonas gingivalis ATCC 33277 |
| Candida parapsilosis | Porphyromonas uenonis |
| Candida sojae | Prevotella amnii |
| Candida viswanathii | Prevotella bergensis DSM 17361 |
| candidate division TM7 single cell isolate TM7a | Prevotella bivia |
| Capnocytophaga gingivalis ATCC 33624 | Prevotella bryantii B14 |
| Capnocytophaga ochracea DSM 7271 | Prevotella buccae D17 |
| Capnocytophaga sputigena ATCC 33612 | Prevotella buccalis |
| Catonella morbi | Prevotella copri |
| Chlamydia trachomatis | Prevotella corporis |
| Clostridioides difficile M120 | Prevotella denticola |
| Clostridioides difficile M68 | Prevotella disiens |
| Clostridium bolteae ATCC BAA 613 | Prevotella marshii DSM 16973 = JCM 13450 |
| Clostridium botulinum D str. 1873 | Prevotella melaninogenica |
| Clostridium leptum DSM 753 | Prevotella melaninogenica D18 |
| Clostridium perfringens C str. JGS1495 | Prevotella oris C735 |
| Clostridium scindens ATCC 35704 | Prevotella oris F0302 |
| Clostridium sp. 7_2_42FAA | Prevotella ruminicola 23 |
| Clostridium sp. M62 1 | Prevotella sp. oral taxon 299 str. F0039 |
| Clostridium sp. SS2/1 | Prevotella sp. oral taxon 317 str. F0108 |
| Collinsella aerofaciens | Prevotella sp. oral taxon 472 str. F0295 |
| Coprinus comatus | Prevotella stercorea |
| Coprococcus comes | Prevotella timonensis |
| Corynebacterium amycolatum | Prevotella veroralis F0319 |
| Corynebacterium aurimucosum | Prevotellamassilia timonensis |
| Corynebacterium glucuronolyticum | Primate T-lymphotropic virus 1 |
| Corynebacterium jeikeium | Primate T-lymphotropic virus 2 |
| Corynebacterium kroppenstedtii DSM 44385 | Propionimicrobium lymphophilum |
| Corynebacterium matruchotil ATCC 33806 | Pseudoflavonifractor capillosus ATCC 29799 |
| Corynebacterium pseudogenitalium ATCC 33035 | Pseudomonas entomophila L48 |
| Corynebacterium striatum ATCC 6940 | Pseudomonas fluorescens Pf0-1 |
| Corynebacterium tuberculostearicum SK141 | Pseudomonas fluorescens SBW25 |
| Corynebacterium urealyticum | Pseudomonas protegens Pf-5 |
| Corynebacterium vitaeruminis | Pseudomonas putida GB-1 |
| Cryptobacterium curtum DSM 15641 | Pseudomonas putida KT2440 |
| Cutibacterium acnes J139 | Pseudomonas putida W619 |
| Cutibacterium acnes J165 | Pseudomonas sp. UK4 |
| Cutibacterium acnes SK137 | Raoultella planticola |
| Cutibacterium acnes SK187 | Roseburia faecis |
| Dialister invisus DSM 15470 | Roseburia intestinalis L1-82 |
| Dialister micraerophilus | Roseburia inulinivorans DSM 16841 |
| Dorea formicigenerans ATCC 27755 | Rothia dentocariosa ATCC 17931 |
| Dorea longicatena DSM 13814 | Rothia dentocariosa M567 |
| Enhydrobacter aerosaccus SK60 | Rothia mucilaginosa ATCC 25296 |
| Entamoeba histolytica | Rothia mucilaginosa DY 18 |
| Enterococcus durans | Ruminococcus albus 8 |
| Enterococcus faecalis | Ruminococcus lactaris ATCC 29176 |
| Enterococcus faecium | Ruminococcus sp. 5_1_39BFAA |
| Enterococcus hirae | Ruminococcus torques ATCC 27756 |
| Enterococcus mundtii | Saccharomyces pastorianus |
| Enterococcus raffinosus | Sarcoptes scabiei |
| Enterococcus ratti | Schistosoma haematobium |
| Enterococcus rivorum | Shuttleworthia satelles DSM 14600 |
| Enterococcus thailandicus | Slackia exigua ATCC 700122 |
| Enterococcus villorum | Sneathia amnii |
| Escherichia coli | Sneathia sanguinegens |
| Eubacterium eligens ATCC 27750 | Solobacterium moorei |
| Eubacterium rectale ATCC 33656 | Sphingobium japonicum UT26S |
| Eubacterium siraeum DSM 15702 | Sphingomonas sp. SKA58 |
| Eubacterium ventriosum ATCC 27560 | Sphingopyxis alaskensis RB2256 |
| Eubacterium yurii subsp margaretiae ATCC 43715 | Staphylococcus aureus |
| Faecalibacterium prausnitzii | Staphylococcus capitis SK14 |
| Faecalibacterium prausnitzii A2-165 | Staphylococcus caprae |
| Fenollaria massiliensis | Staphylococcus devriesei |
| Filifactor alocis ATCC 35896 | Staphylococcus epidermidis |
| Finegoldia magna | Staphylococcus epidermidis BCM HMP0060 |
| Finegoldia magna ACS 171 V Col3 | Staphylococcus epidermidis M23864:W2grey |
| Finegoldia magna ATCC 53516 | Staphylococcus epidermidis RP62A |
| Fusobacterium equinum | Staphylococcus epidermidis SK135 |
| Fusobacterium gonidiaformans | Staphylococcus epidermidis W23144 |
| Fusobacterium gonidiaformans ATCC 25563 | Staphylococcus haemolyticus |
| Fusobacterium mortiferum ATCC 9817 | Staphylococcus hominis |
| Fusobacterium nucleatum | Staphylococcus petrasii |
| Fusobacterium nucleatum subsp animalis D11 | Staphylococcus saprophyticus |
| Fusobacterium nucleatum subsp nucleatum ATCC | Staphylococcus warneri |
| 23726 | |
| Fusobacterium nucleatum subsp polymorphum | Stenotrophomonas maltophilia K279a |
| ATCC 10953 | |
| Fusobacterium nucleatum subsp vincentii 4_1_13 | Streptobacillus moniliformis DSM 12112 |
| Fusobacterium nucleatum subsp vincentii ATCC | Streptococcus agalactiae |
| 49256 | |
| Fusobacterium nucleatum subsp. animalis 3_1_33 | Streptococcus agalactiae 18RS21 |
| Fusobacterium nucleatum subsp. animalis 7_1 | Streptococcus agalactiae CJB111 |
| Fusobacterium nucleatum subsp. vincentii 3_1_27 | Streptococcus agalactiae COH1 |
| Fusobacterium nucleatum subsp. vincentii | Streptococcus agalactiae NEM316 |
| 3_1_36A2 | |
| Fusobacterium periodonticum 1_1_41FAA | Streptococcus anginosus |
| Fusobacterium periodonticum 2_1_31 | Streptococcus cristatus |
| Fusobacterium periodonticum ATCC 33693 | Streptococcus equi subsp equi 4047 |
| Gardnerella vaginalis | Streptococcus equinus |
| Gardnerella vaginalis 5-1 | Streptococcus gallolyticus |
| Gardnerella vaginalis AMD | Streptococcus gordonii str. Challis substr CH1 |
| Gardnerella vaginalis ATCC 14019 | Streptococcus infantarius |
| Gemella asaccharolytica | Streptococcus intermedius |
| Gemella haemolysans | Streptococcus lutetiensis |
| Gemella sanguinis | Streptococcus macedonicus |
| Giardia intestinalis | Streptococcus mitis |
| Granulicatella elegans | Streptococcus oralis subsp dentisani |
| Haemophilus ducreyi 35000HP | Streptococcus oralis subsp tigurinus |
| Haemophilus parainfluenzae | Streptococcus parasanguinis |
| Helicobacter pylori B128 | Streptococcus pasteurianus |
| Helicobacter pylori B8 | Streptococcus pneumoniae str. Canada MDR 19A |
| Hepacivirus C | Streptococcus pneumoniae str. Canada MDR 19F |
| Hepatitis B virus | Streptococcus pneumoniae TCH8431/19A |
| Herbaspirillum seropedicae SmR1 | Streptococcus pseudopneumoniae |
| Human alphaherpesvirus 1 | Streptococcus pyogenes |
| Human alphaherpesvirus 2 | Streptococcus salivarius |
| Human immunodeficiency virus | Streptococcus sanguinis |
| Hungatella hathewayi DSM 13479 | Streptococcus sp. 2_1_36FAA |
| Jonquetella anthropi | Streptococcus sp. M143 |
| Lactobacillus acidophilus | Streptococcus suis BM407 |
| Lactobacillus acidophilus ATCC 4796 | Subdoligranulum variabile DSM 15176 |
| Lactobacillus agilis | Talaromyces marneffei |
| Lactobacillus amylolyticus DSM 11664 | Treponema pallidum |
| Lactobacillus amylovorus | Trichomonas vaginalis |
| Lactobacillus antri DSM 16041 | Tyzzerella nexilis DSM 1787 |
| Lactobacillus casei | Ureaplasma parvum |
| Lactobacillus coleohominis | Ureaplasma parvum serovar 6 str. ATCC 27818 |
| Lactobacillus crispatus | Ureaplasma urealyticum |
| Lactobacillus crispatus 125-2-CHN | Ureaplasma urealyticum serovar 9 str. ATCC 33175 |
| Lactobacillus crispatus 214-1 | Vanderwaltozyma polyspora |
| Lactobacillus crispatus JV-V01 | Varibaculum cambriense |
| Lactobacillus crispatus MV-1A-US | Veillonella atypica |
| Lactobacillus crispatus MV-3A-US | Veillonella dispar |
| Lactobacillus curieae | Veillonella montpellierensis |
| Lactobacillus delbrueckii | Veillonella parvula ATCC 17745 |
| Lactobacillus delbrueckii subsp bulgaricus ATCC | Veillonella parvula DSM 2008 |
| 11842 | |
| Lactobacillus delbrueckii subsp bulgaricus | Veillonella seminalis |
| PB2003/044-T3-4 | |
| Lactobacillus fermentum | Veillonella sp. 3_1_44 |
| Lactobacillus fermentum 28 3-CHN | Veillonella sp. 6_1_27 |
| Weissella cibaria | Weissella confusa |

The complex microbiome as used herein is preferably isolated from the gut, skin, bladder, skin, mouth, nose, ears, lungs or the cervicovaginal area, preferably the cervicovaginal area. In preferred embodiment, the invention relates to the profiling of the cervicovaginal area.

In preferred embodiments of the invention, multiplex mRNA sequencing is performed using at least one MIP selected from the group listed in Table II.

TABLE II
List of designed molecular inversion probes.

| | |
|---|---|
| smMIP1 | AGTCACTATCCAGACCAAAGCGCCCNNNNNNNNCTTCAGCTTCCCGATATCCGACGGTAGTGTCAG |
| | ATGGAAAAGATGGTCT |
| smMIP2 | GGATGGTGGTGCATGGCCGTTCTTAGNNNNNNNNCTTCAGCTTCCCGATATCCGACGGTAGTGTAG |
| | AAATTGACGGAAGGGCA |
| smMIP3 | TGCGCCTACAAGCGGTCGGAGCTNNNNNNNNCTTCAGCTTCCCGATATCCGACGGTAGTGTTTCTG |
| | CGTGAATCTGCCGGG |
| smMIP4 | GCTTAACACATGCAAGTCGNNNNNNNNCTTCAGCTTCCCGATATCCGACGGTAGTGTTAATAGGTAT |
| | TTGAATAAGGT |
| smMIP5 | TGACCTTATTCAAATACCTANNNNNNNNCTTCAGCTTCCCGATATCCGACGGTAGTGTTCGACTTGC |
| | ATGTGTTAAGC |
| smMIP6 | CGTAACAAGGTAGCCGTACCGGAANNNNNNNNCTTCAGCTTCCCGATATCCGACGGTAGTGTAATG |
| | CGTTCCCGGGCCTTGTA |
| smMIP7 | GTAGTCATATGCTTGTCTCNNNNNNNNCTTCAGCTTCCCGATATCCGACGGTAGTGTTGGCTAGTTG |
| | TAGAGAGTAGTAAAA |
| smMIP8 | ACGGCCCACCAAAGCGACGATCAGTAGNNNNNNNNCTTCAGCTTCCCGATATCCGACGGTAGTGTC |
| | TGGGGGATAACAGTTAG |
| smMIP9 | CAAGGTTTCCGTAGGTGAACCNNNNNNNNCTTCAGCTTCCCGATATCCGACGGTAGTGTACCGCCC |
| | GTCGCTACTACCG |
| smMIP10 | GGTGGTGCATGGCTGTCGTCNNNNNNNNCTTCAGCTTCCCGATATCCGACGGTAGTGTAATTGACG |
| | GGGCCCCGCACAAGC |
| smMIP11 | CGGCGGCCGTAACTATAACGGTNNNNNNNNCTTCAGCTTCCCGATATCCGACGGTAGTGTCCCAGG |
| | CAACTGTTTATCAAAA |
| smMIP12 | GTGGGCAGTTTGACTGGGGCGGTNNNNNNNNCTTCAGCTTCCCGATATCCGACGGTAGTGTTGTAC |
| | AGGATAGGTGGGAGA |
| smMIP13 | GCAAGGTTGAAACTCAAAGGAATTGACGNNNNNNNNCTTCAGCTTCCCGATATCCGACGGTAGTGT |
| | TAGCAAACAGGATTAGA |
| smMIP14 | GATAGGTTGGGGGTGTACGCGCAGTAATNNNNNNNNCTTCAGCTTCCCGATATCCGACGGTAGTGT |
| | GTTGTCACGCCAGTGG |
| smMIP15 | CACTGTTTTGATTTTTTACACCTTGNNNNNNNNCTTCAGCTTCCCGATATCCGACGGTAGTGTTTACG |
| | CGTTACTCACCCGTC |
| smMIP16 | GGTTTTCTGCGTTCAGCCTGAGAAGGGGGNNNNNNNNCTTCAGCTTCCCGATATCCGACGGTAGTG |
| | TATGGGGCAAAAGACGT |
| smMIP17 | GTGGTTATCCTGCGTTGATGCCTNNNNNNNNCTTCAGCTTCCCGATATCCGACGGTAGTGTCCACAA |
| | GAACACATCATACAA |
| smMIP18 | GTAAGGTGCAGAAAGAATATGCANNNNNNNNCTTCAGCTTCCCGATATCCGACGGTAGTGTCTCTGT |
| | GTTAGTTTAAAGTGCA |
| smMIP19 | GGGGAGTACGGCCGCAAGGTTGAAACTNNNNNNNNCTTCAGCTTCCCGATATCCGACGGTAGTGTG |
| | CGAAAGCGTGGGTAG |
| smMIP20 | AATGAAAACGTCCTTGGCAAATGCNNNNNNNNCTTCAGCTTCCCGATATCCGACGGTAGTGTTGATT |
| | TCTCGTAAGGTGCCG |
| smMIP21 | GCACGAGCTGACGACAACCANNNNNNNNCTTCAGCTTCCCGATATCCGACGGTAGTGTTGCTGATT |
| | TGACGTCATCCCCACCT |
| smMIP22 | GGGGGATTAGCTCAGTTGGCTAGANNNNNNNNCTTCAGCTTCCCGATATCCGACGGTAGTGTGGAA |
| | GGTGCGGCTGGA |
| smMIP23 | CCCTGTCACAGAACTGCCGCNNNNNNNNCTTCAGCTTCCCGATATCCGACGGTAGTGTTCCAAAGG |
| | AACTCCTACCTTACGCC |
| smMIP24 | GACTCTAAATATTTAATCAAATACCTNNNNNNNNCTTCAGCTTCCCGATATCCGACGGTAGTGTCCTT |
| | CAACAGGACATCA |
| smMIP25 | TGGAACAGGACGTCATAGAGNNNNNNNNCTTCAGCTTCCCGATATCCGACGGTAGTGTAAACCAAC |
| | CGGGATTGCCTT |
| smMIP26 | GCATGATGATTTGACGTCATCCCNNNNNNNNCTTCAGCTTCCCGATATCCGACGGTAGTGTCTTCAT |
| | GTAGTCGAGTTGCAGA |
| smMIP27 | GGAGAGCGCCTGCTTTGCACGCAGGANNNNNNNNCTTCAGCTTCCCGATATCCGACGGTAGTGTAA |
| | GTCGTAACAAGGTAGCC |
| smMIP28 | CAGCGTTCGTCCTGAGCCAGGATCAAACTNNNNNNNNCTTCAGCTTCCCGATATCCGACGGTAGTG |
| | TTCCAATAGTTATCCCC |
| smMIP29 | GATACATAGCCGACCTGAGAGGGTGATCGNNNNNNNNCTTCAGCTTCCCGATATCCGACGGTAGTG |
| | TAGCTAATACCGCATAA |
| smMIP30 | TTTGATCCTGGCTCAGGACGAACGCNNNNNNNNCTTCAGCTTCCCGATATCCGACGGTAGTGTTTG |
| | AAAACTGAACAAGAC |
| smMIP31 | GCCACGGCTAACTACGTGCCAGCAGNNNNNNNNCTTCAGCTTCCCGATATCCGACGGTAGTGTGGA |
| | CGAAAGTCTGACGGAG |

In certain embodiments of the invention, the method comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 MIPs from the from the group listed in table II.

In certain embodiments of the invention, the genes of interest are selected from the group of genes that encode for enzymes that are involved in tyrosine metabolism, tryptophan metabolism, bile acid metabolism, fatty acid metabolism, amino acid metabolism, 16S rRNA genes, 23S rRNA genes and genes encoding toxins. These genes, which are involved in health and disease and can determine the response to medicines, are known to the person skilled in the art.

In certain embodiments, smMIPs are designed to detect regions of interest in genes of interest from a microbial genus, a microbial species or a microbial strain of interest, and selected for the potential of such smMIP to specifically reveal the identity of such microbial genus, species or strain.

In certain embodiments, the method of the invention is for identifying the relationships between microbial genuses, species or strains and the host environment in which these microbial genuses, species or strains occur.

In certain embodiments, the method of the invention is for identifying microbial compositions and functions in the mouth, airways, gut, cervix, urinary bladder, skin, ears and eyes that are diagnostic and prognostic for disease, including but not limited to tooth decay, head and neck cancer, pneumonia, eczema, lychen sclerosis, bladder cancer, bladder infection, cervicovaginal malignancies or disorders such as cervical intraepithelial neoplasia and cervical cancer, inflammatory bowel disease, colon adenomas, and colon cancer.

In certain embodiments, the method of the invention relates to a method of identifying a candidate diet or therapy for treatment of a disease or disorder including but not limited to tooth decay, head and neck cancer, pneumonia, eczema, lychen sclerosis, bladder cancer, bladder infection, cervicovaginal malignancies or disorders such as cervical intraepithelial neoplasia and cervical cancer, inflammatory bowel disease, colon adenomas, colon cancer and neurological diseases including but not limited to Alzheimers disease, Multiple Sclerosis and Parkinson disease.

In another aspect, the invention provides for a molecular inversion probe selected from the group listed in table II.

In yet another aspect, the invention provides for a set of molecular inversion probes comprising at least two MIPs selected from the group listed in table II.

In yet another aspect, the invention relates to a method of identifying a candidate treatment for a disease or disorder as defined herein in a subject in need thereof, comprising:
providing a sample from the subject and subjecting the sample to the method as defined herein in the first aspect; and
identifying a treatment that is beneficially associated with the molecular profile of the subject, thereby identifying the candidate treatment.

In certain embodiments the cervicovaginal malignancy or disorder may be a cancer of the female genitourinary system. In yet another embodiment the cervicovaginal disorder may be an infection of the female genitourinary system.

In yet another aspect, the invention provides for a method of detecting the presence or absence of a target nucleic acid in a complex microbiome sample, preferably from the cervicovaginal area, wherein the method comprises:
a) contacting the sample with at least one molecular inversion probe (MIP), wherein said MIP comprises a first hybridization arm comprising a first sequence complementary to a first region in the target nucleic acid of interest, a second hybridization arm comprising a second sequence complementary to second region in the target nucleic acid of interest and a detectable moiety,
b) extending the extension arm with a DNA polymerase and ligating the extended MIP ends that are hybridized to complementary targets, to the ligation arm of said MIPs to form circularized MIPs,
c) purifying the circularized MIP;
d) amplifying the purified circularized MIP, preferably by PCR;
e) optionally, purifying the amplified product containing the MIP sequence
f) subjecting the amplified product to next generation sequencing; and
g) detecting the presence or absence of a target nucleic acid in the sample by detecting the presence or absence of the corresponding amplified MIP sequence,
   wherein at least one MIP is selected from the group listed in table II.

In the embodiments herein, the samples may be any sample from a subject that is useful for a method herein, such a sample from a bodily fluid or excrement, such as a cervicovaginal swab or a faeces sample. A sample is preferably an ex vivo sample.

### Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptides obtainable by expression of the genes present in SEQ ID NO: 1 containing the nucleic acid sequences coding for the polypeptides should prevail.

All patent and literature references cited in the present specification are hereby incorporated by reference in their entirety.

### EXAMPLES

### Methods

### smMIP Design and Targeted Sequencing

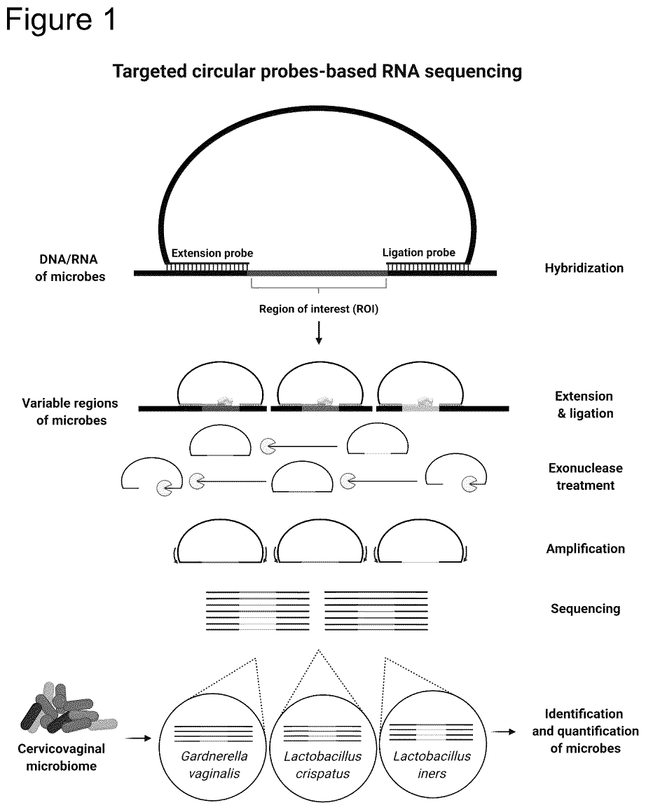

We compiled a list of 434 previously identified microbes that have been recognized as significant in the cervicovaginal environment by recent literature and the Human Vaginal Microbiome Project (Additional File 1) [37, 38]. The genome sequences were initially retrieved from the National Center for Biotechnology Information (NCBI, [39]) using Biomartr [40]. Sequences from small ribosomal subunit (SSU) and large ribosomal subunit (LSU) rRNA genes were selected and extracted using Biopython [41] and BEDTools [42], respectively [23, 43]. smMIPs against SSU and LSU rRNA genes were designed in MIPgen [35]. We selected smMIPs with homologous hybridization arms and dissimilar regions of interest (ROIs) and included a random octanucleotide UMI in the smMIP backbone. Next, we compared the selected ROI sequences with the corresponding rRNA sequences within the SILVA rRNA database. Only sequences that were 100% full length consistent with this database were regarded as fit for annotation [44]. Subsequently, MegaBLAST and the Burrows-Wheeler Aligner (BWA) were combined to validate in silico the specificity of smMIPs in discriminating species [45, 46]. Thereafter, a greedy algorithm was implemented to validate the potential of a smMIP in identifying as many species at once as possible based on ROIs sequences. This validation resulted in the selection of 30 smMIPs targeting the 434 microbes and pathogens (Table 3). All smMIPs were validated on a dataset composed of genomes and annotations from species isolated from cervical smears (Supplementary FIG. 1). Then, to standardize species' detection and reduce the chance of false positive annotation, we considered only species that were identified with two or more reactive smMIPs. This filtering resulted in the final selection of our targets consisting of 107 genera and 321 species that represent our cervicovaginal microbiome panel (CVMP), including bacteria, fungi, and parasites (Additional File 3). CiRNAseq was performed as previously described [30, 34]. For the sequencing of individual species, 10 ng of microbe DNA was analyzed. Analyses of cervicovaginal samples were performed on ˜50 ng of cDNA/DNA generated according to standard protocols (see Study participants and samples). Following capture hybridization and probe circularization and purification, circularized probes were subjected to PCR with barcoded Illumina primers. After purification of the correct-size amplicons, quality control, and quantification as previously described [34], a 4 nM library was sequenced on the Illumina Nextseq500 platform (Illumina, San Diego, CA) at the Radboudumc sequencing facility.

TABLE III
List of smMIPs and sequences used for CiRNAseq profiling of the cervicovaginal
microbiome

| ID | Extension probe | Ligation probe |
|---|---|---|
| smMIP1 | CAGATGGAAAAGATGGTCT | AGTCACTATCCAGACCAAAGCGCCC |
| smMIP2 | AGAAATTGACGGAAGGGCA | GGATGGTGGTGCATGGCCGTTCTTAG |
| smMIP3 | TTCTGCGTGAATCTGCCGGG | TGCGCCTACAAGCGGTCGGAGCT |
| SmMIP4 | AATGCGTTCCCGGGCCTTGTA | CGTAACAAGGTAGCCGTACCGGAA |
| SmMIP5 | TGGCTAGTTGTAGAGAGTAGTAAAA | GTAGTCATATGCTTGTCTC |
| smMIP6 | CTGGGGGATAACAGTTAG | ACGGCCCACCAAAGCGACGATCAGTAG |
| smMIP7 | ACCGCCCGTCGCTACTACCG | CAAGGTTTCCGTAGGTGAACC |
| smMIP8 | AATTGACGGGGCCCCGCACAAGC | GGTGGTGCATGGCTGTCGTC |
| smMIP9 | CCCAGGCAACTGTTTATCAAAA | CGGCGGCCGTAACTATAACGGT |
| smMIP10 | TGTACAGGATAGGTGGGAGA | GTGGGCAGTTTGACTGGGGCGGT |
| smMIP11 | TAGCAAACAGGATTAGA | GCAAGGTTGAAACTCAAAGGAATTGACG |
| smMIP12 | GTTGTCACGCCAGTGG | GATAGGTTGGGGGTGTACGCGCAGTAAT |
| SMMIP13 | TTACGCGTTACTCACCCGTC | CACTGTTTTGATTTTTTACACCTTG |
| smMIP14 | ATGGGGCAAAAGACGT | GGTTTTCTGCGTTCAGCCTGAGAAGGGGG |
| SmMIP15 | CCACAAGAACACATCATACAA | GTGGTTATCCTGCGTTGATGCCT |
| SmMIP16 | CTCTGTGTTAGTTTAAAGTGCA | GTAAGGTGCAGAAAGAATATGCA |
| smMIP17 | GCGAAAGCGTGGGTAG | GGGGAGTACGGCCGCAAGGTTGAAACT |
| smMIP18 | TGATTTCTCGTAAGGTGCCG | AATGAAAACGTCCTTGGCAAATGC |
| smMIP19 | TGCTGATTTGACGTCATCCCCACCT | GCACGAGCTGACGACAACCA |
| smMIP20 | GGAAGGTGCGGCTGGA | GGGGGATTAGCTCAGTTGGCTAGA |
| smMIP21 | TCCAAAGGAACTCCTACCTTACGCC | CCCTGTCACAGAACTGCCGC |
| smMIP22 | CCTTCAACAGGACATCA | GACTCTAAATATTTAATCAAATACCT |
| smMIP23 | AAACCAACCGGGATTGCCTT | TGGAACAGGACGTCATAGAG |
| smMIP24 | CTTCATGTAGTCGAGTTGCAGA | GCATGATGATTTGACGTCATCCC |
| smMIP25 | AAGTCGTAACAAGGTAGCC | GGAGAGCGCCTGCTTTGCACGCAGGA |
| smMIP26 | TCCAATAGTTATCCCC | CAGCGTTCGTCCTGAGCCAGGATCAAACT |
| smMIP27 | AGCTAATACCGCATAA | GATACATAGCCGACCTGAGAGGGTGATCG |
| smMIP28 | TTGAAAACTGAACAAGAC | TTTGATCCTGGCTCAGGACGAACGC |
| smMIP29 | GGACGAAAGTCTGACGGAG | GCCACGGCTAACTACGTGCCAGCAG |
| smMIP30 | TCGACTTGCATGTGTTAAGC | TGACCTTATTCAAATACCTA |

### CiRNAseq Output Analysis

Reads were mapped against reference ROIs within our CVMP using the SeqNext module of JSI Sequence Pilot version 4.2.2 build 502 (JSI Medical Systems, Ettenheim, Germany). The settings for read processing were a minimum of 50% matching bases, a maximum of 15% mismatches, and a minimum of 50% consecutive bases without a mismatch between them; for read assigning, the threshold was a minimum of 95% of homologous bases with the ROIs. All identical PCR products were reduced to one consensus read (unique read counts, URC) using the UMI. We set an arbitrary threshold of at least 1000 URC from all smMIPs combined in an individual sample, below which we considered an output non-interpretable [47]. For microbial annotation, species with two reactive smMIPs were annotated when 100% of the specific set of smMIPs had URC. Species with three or more reactive smMIPs were annotated when more than 50% of their specific set of smMIPs had URC using a custom R script. For analyses where isolates from our CVMP were not considered, the URC for each isolate were summed to represent the bacterium at the species level. To define relative abundances, microbial species URC was divided by the total URC of all microbes annotated in the sample. For establishing microbial diversity, URC was turned to 1 and 0, indicating the presence or absence of microbes, respectively.

### 16S rRNA Gene Amplification and Sequencing

Residual material from ten hrHPV positive cervical smears in PreservCyt solution, randomly obtained from the Dutch population-based cervical cancer screening program (CCSP) with approval from the regional institutional review board and the National Institute for Public Health and Environment (No. 2014-1295), was initially pelleted by centrifugation. Pellets were suspended in 1 ml DNA/RNA shield buffer (Zymo, cat. no. R1104). DNA was extracted according to standard protocols and processed by BaseClear B.V. (Leiden, the Netherlands) for microbiome profiling using the primers 357F (5′-CCTACGGGAGGCAGCAG-3′) and 802RV2 (5′-TACNVGGGTATCTAAKCC-3′) that target the V3 and V4 variable regions of the 16S rRNA gene [28]. PCR protocol was as follows: 2 m 95° C. hot start; 35 cycles of 20 s 95° C., 10 s 61° C., 15 s 70° C.; 10 m 70° C. The libraries were barcoded, multiplexed, and sequenced on an Illumina MiSeq machine with paired-end 300 cycles protocol and indexing by BaseClear [48]. Illumina sequencing data were quality checked and demultiplexed by BaseClear standards, and FASTQ files were generated.

### 16S rRNA Gene Sequencing Data Analysis

From the FASTQ files, forward and reverse reads were pairwise assembled with PEAR (v0.9.10, [49]) in default settings. For the generation of the 16S-derived taxa-to-sample compositional matrix, a customized Python workflow based on Quantitative Insights Into Microbial

Ecology (QIIME v1.8, [50]) was adopted (http://qiime.org). Relative abundances per sample were calculated with QIIME default settings, where the reads per taxon were divided by the total number of bacterial reads for that sample. Following the comparison analyses with CiRNAseq, a misassigning of reads from genus Gardnerella to Bifidobacterium by QIIME was determined using BLAST to the original DNA sequences. Subsequently, absolute and relative abundances were manually corrected.

### In Vitro Validation of Sequencing Targets

To test in vitro the specificity and resolution of CiRNAseq, we used 12 bacteria species listed in Supplementary Table 1, obtained from the Medical Microbiology Department, Radboudumc, Nijmegen, the Netherlands. Bacteria were grown in appropriate culture media. Following growth, their genomic DNA was extracted using DNA and Viral Small volume kit (Roche, cat. no. 6543588001). PCR and Sanger sequencing was performed to validate species identification. Water was used as the negative control. For CiRNAseq, we prepared a concentration of 1.5 ng/μL from each microbes' DNA in a final volume of 40 μL.

### In Vitro Validation of RNA Testing and Quantification

To assess the capacity of CiRNAseq to quantify and analyze microbial RNA, an Escherichia coli (E. coli; ATCC 25922) culture in stationary phase was inoculated at 5% in BHI medium and incubated at 37° C. on a shaking platform at 100 rpm for 48 hours. Optical density (OD630) was measured every hour, and 1 ml aliquots were taken after each measurement, pelleted, and stored for nucleic acid isolation. After 26.5 hours of culture, an aliquot was taken for autoclaving. A second aliquot was treated with 0.75 ml of cefoxitin (1 mg/ml), followed by further growth for an additional 20 hours (Supplementary Table 2). Nucleic acids were isolated from all aliquots using the MagNA Pure kit (Roche, cat. no. 03730964001). RNA concentrations (ng/ml) were measured using NanoDrop 2000 (Thermo Scientific). After treatment with DNAase, RNA was processed to cDNA for CiRNAseq analysis.

### Study Participants and Samples

For this study, a total of 102 cervical smears in PreservCyt were collected from women participating in the Dutch CCSP, which were received and processed at Radboudumc (Nijmegen, the Netherlands). Women participating in the CCSP were informed that residual material could be used for anonymous research and had the opportunity to opt-out. Only residual material from women who did not opt-out was included. The histological follow-up outcomes were obtained from the nationwide network and registry of histo- and cytopathology in the Netherlands (PALGA; Houten, the Netherlands). hrHPV identification was performed as previously described [34]. All methods were performed following the institutional guidelines for using human samples. One set of ten hrHPV positive smears was used for the comparative analyses with 16S rRNA-seq. DNA from these samples was isolated from 1 ml of residual material using DNA and Viral Small volume kit (Roche, cat. no. 6543588001) and subjected to CiRNAseq. The cohort of the remaining 92 cervical smears consisted of 46 hrHPV positive samples of women with confirmed high-grade cervical intraepithelial neoplasia (CIN2+) and 46 hrHPV DNA negative smears. Five ml of each cervical cell suspension was centrifuged for 5 min at 2,500×g, and the pellet dissolved in 1 ml of Trizol reagent (Thermo Scientific). RNA was isolated through standard procedures and dissolved in 20 μl nuclease-free water. We routinely processed a maximum of 2 μg of RNA for DNase treatment and cDNA generation, using SuperscriptII (Thermo) as previously described [34].

### Statistical Analyses

Analyses with our CVMP were performed using ClustVis [51]. For the microbiome shift clustering analysis, the settings were as follows: clustering distance for columns: Canberra [52, 53]; clustering method: Ward (unsquared distances); row scaling: Pareto scaling [54]. Canberra distance normalizes the absolute difference in abundance of each taxon, allowing comparison of minor taxa. A shorter Canberra distance indicates greater similarity.

Linear discriminant analysis (LDA) effect size was performed using the LEfSe tool [55]. LEfSe combines standard tests for statistical significance (Kruskal-Wallis test and pairwise Wilcoxon test) with LDA for feature selection. Alpha value for the factorial Kruskal-Wallis test was 0.05. Threshold on the logarithmic LDA score for discriminative features was 2.0 [55].

Microsoft Excel 2016® and GraphPad Prism v9.0.0 (GraphPad Software, Inc., USA) were used to analyze datasets and determine species richness, Shannon's diversity index, and Pearson's r correlations. The statistical significance of differences in microbial richness, diversity, and relative abundance were calculated using GraphPad with a Mann-Whitney test to obtain the p-value. Significant differences between groups are denoted by *p<0.05, **p<0.01, ***p<0.001, or ****p<0.0001.

### Results

Here we tested the hypothesis that CiRNAseq can be used for high-resolution microbiome profiling. The technology, summarized in FIG. 1, uses probes with homologous hybridization arms with high specificity for ribosomal RNA, that flank heterologous regions of interest. With bioinformatic analyses, we selected 30 smMIPs that combined can detect 107 genera and 321 species relevant in the cervicovaginal environment (FIG. 1 and Supplementary 1) [37, 38]. By comparing these ROIs with a reference database, this method assigns URC to microbes of interest. Because we require that at least two different ROIs must be detected in a microbe, the CiRNAseq pipeline ensures a robust species-level annotation of the microbiome (FIG. 1).

Thirty different single-molecule molecular inversion probes (smMIPs) are available to hybridize to the 16S rRNA gene of microbes identified as part of the cervicovaginal microbiome. In the cervicovaginal microbiome, hundreds of microbe species can be detected, playing a role in health and disease. smMIPs were selected based on extension and ligation arms that are shared between species and flanking hypervariable regions of interest (ROI) that are unique per species. After smMIP hybridization and filling in the ROI gaps, followed by ligation, the library of circularized smMIPs is PCR amplified with barcoded Illumina primers and sequenced. All collected ROI sequences in a sample are then compared to a reference database containing reference ROIs from all microbial species of interest. Based on a combination of two or more ROIs, the microbiome can be annotated in high-resolution. The assay is made quantitative by incorporating a unique molecule identifier (UMI), which eliminated PCR amplification bias.

We performed in vitro and in silico validations to demonstrate the potential of CiRNAseq for high throughput sequencing of the microbiome and compared this new method to 16S rRNA-seq. We designed a dedicated CiRNAseq test to study the CVM in smears from hrHPV negative women and women with hrHPV-associated CIN2+ lesions. We also validated the specificity, resolution, reproducibility, targeting (DNA/RNA), and quantification abilities of the technology in profiling the CVM.

### CiRNAseq Exhibits High Specificity and Resolution

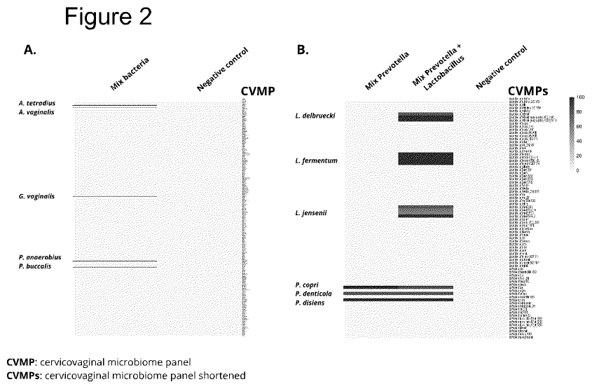

To validate the specificity of CiRNAseq in a mixed microbial environment, we first tested the technique by analyzing a defined mixture of genomic DNA from Anaerococcus tetradius, Anaerococcus vaginalis, Gardnerella vaginalis, Peptostreptococcus anaerobius, and Prevotella buccalis, which are typical for the CVM (FIG. 2A, Supplementary Table 1). Water was used as a negative control. CiRNAseq correctly identified the five input species based on sequence comparison with the reference ROIs and with the restriction that at least 50% of their specific set of smMIPs were reactive. In the negative control, the technique did not yield any data (FIG. 2A). Thus, CiRNAseq can discriminate microbes in a mixed microbial sample with high specificity. Subsequently, we assessed the technique's resolution in detecting microbes at the species level (FIG. 2B). To this end, we prepared a mixed microbial sample consisting of genomic DNA from three species of Prevotella (P. copri, P. denticola, and P. disiens) and added these to a second mixed sample containing DNA from three Lactobacillus species (L. delbruecki, L. fermentum, and L. jensenii). All of these species are commonly found in the CVM. As represented in FIG. 2B, CiRNAseq correctly identified all individual species in all samples. Thus, CiRNAseq is able to distinguish microbes at the species level for this specific mixed microbial sample and application.

### CiRNAseq RNA Quantification Capacity Mirrors Bacterial Growth and Activity

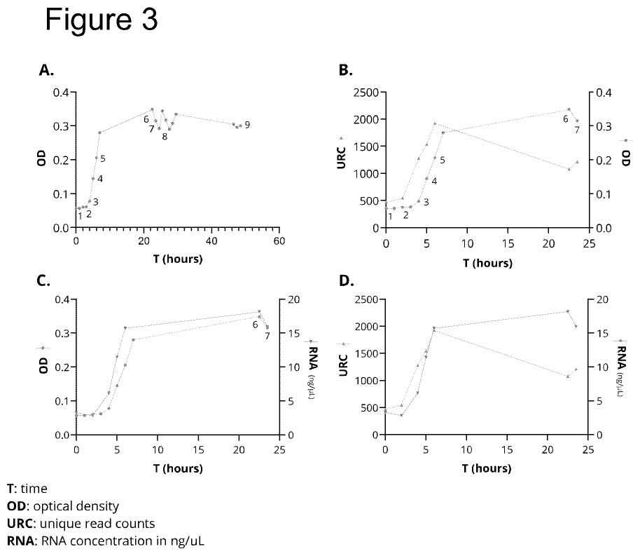

In natural niches such as the CVM, DNA is a very stable molecule, while RNA is rapidly degraded. Therefore, whereas DNA sequencing can reveal the presence of genomic DNA of bacterial species in a sample, RNA sequencing gives information on the activity of such species by identifying which genomic regions are transcribed to RNA [56]. To evaluate the CiRNAseq potential in quantifying active microbes at the RNA level, we examined how the growth of E. coli, a species that can be found in the CVM [57, 58], is reflected in the number of unique read counts (URC) obtained from RNA sequencing. Following the growth of a pure culture of E. coli for 48 hours through OD measurement every hour, we selected nine-time points where the E. coli culture was sampled for RNA isolation, including the bacterial lag, exponential, and stationary phases (FIG. 3A, in orange dots). We also selected two samples that were either autoclaved or treated with an antibiotic. Samples were taken in duplicate and subjected to CiRNAseq to test reproducibility. The mean number of URC achieved in these replicates for the lag and exponential phases is shown in FIG. 3B (green line, first seven-time points) and Supplementary 2B. When comparing the OD of E. coli culture to the mean of URC obtained from sequencing, we found that the values were significantly correlated, particularly from the lag to the exponential phase (p=0.0286) (FIG. 3B). Samples taken from the stationary growth phase had lower URCs, indicating lower ribosomal activity in bacteria from the stationary phase than bacteria from the exponential growth phase.

We also analyzed the RNA concentrations of each aliquot taken for sequencing and compared them to the OD and URC, as shown in FIGS. 3C, 3D. Here we noticed that the isolated total RNA matched the OD of E. coli growth phases (FIG. 3C). Furthermore, we observed that the RNA levels of the samples taken from the stationary phase (time points six and seven) were higher than those from the exponential phase (FIG. 3D), reflecting the accessible RNA for sequencing. As expected, we did not find any URC after autoclaving the sample taken in time point eight, even though the OD and RNA concentration measured prior to autoclaving was similar to the growth phase. Similarly, the sample treated with cefoxitin (antibiotic) had a low number of URC, suggesting inhibition of ribosomal activities. Thus, CiRNAseq can quantify microbes' RNA, mirroring translational activity and growth.

### CiRNAseq Holds a Deeper Sequencing Performance than 16S rRNA-seq

Given that the gold-standard sequencing method for profiling the microbiome is 16S rRNA-seq, we compared both sequencing methodologies. To this purpose, we randomly selected ten hrHPV positive smears, which were simultaneously profiled using CiRNAseq and 16S rRNA-seq at the DNA level.

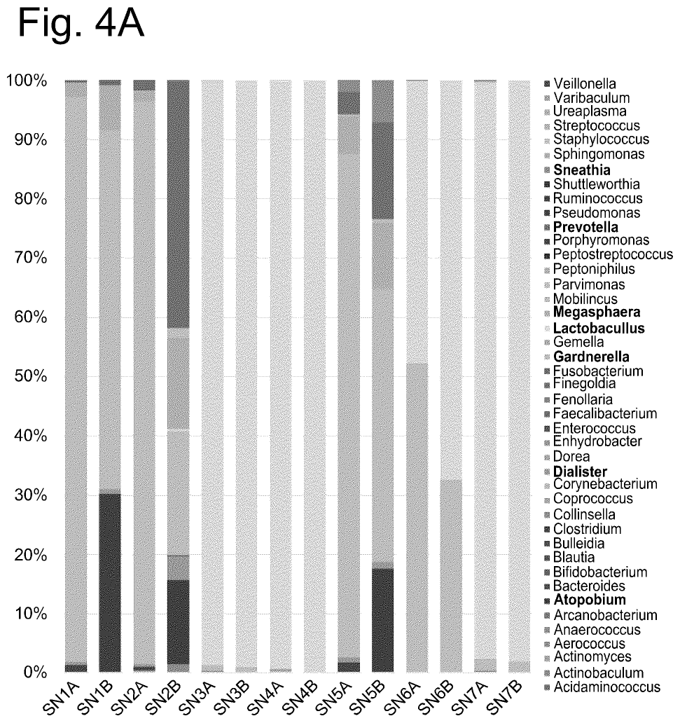

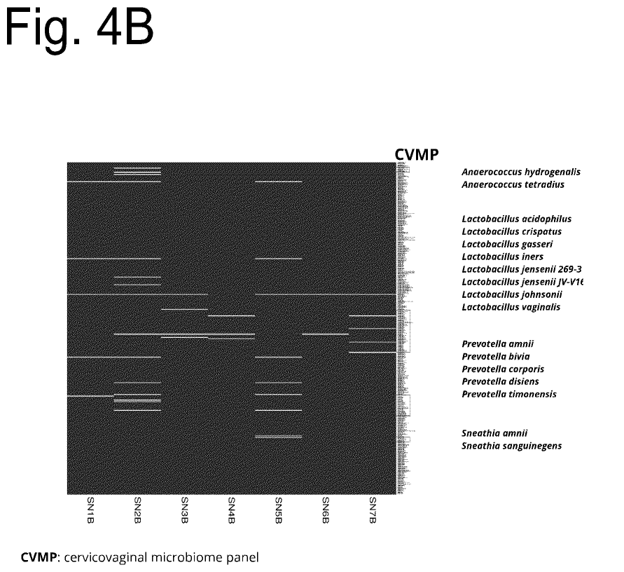

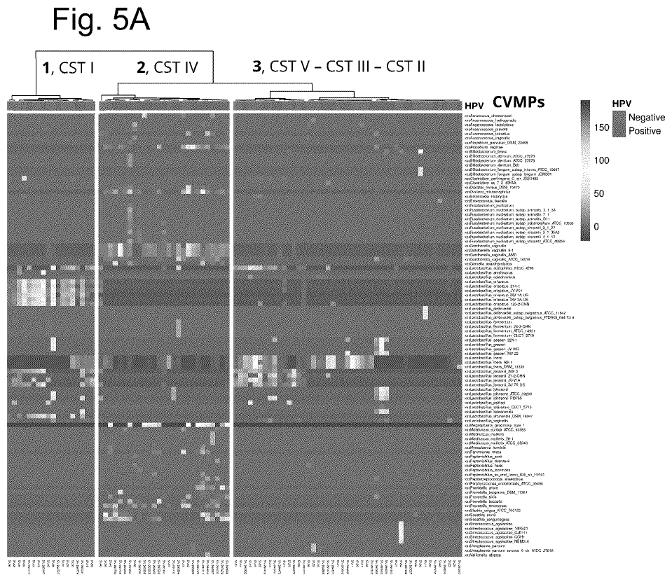

Two out of ten samples had low reads (<2500 reads) with 16S rRNA-seq compared to the rest of the samples (>80000 reads) and were excluded from the analyses. One additional sample had <1000 URC with CiRNAseq and was also excluded from the study. In the remaining seven samples, we determined the relative microbes' abundances. Following 16S rRNA-seq, we focused our analyses on 43 genera that were profiled by 16S rRNA-seq and were also available for microbiome profiling using CiRNAseq (FIG. 4 and Additional File 3). Microbes with relative abundances ≤0.06% were considered non-present in the samples.

The seven remaining samples sequenced with 16S rRNA-seq (SN-A) and CiRNAseq (SN-B) were analyzed, as shown in FIG. 4. Here, we first observed that the relative abundances are highly similar using both techniques (FIG. 4A), suggesting that CiRNAseq and 16S rRNA-seq have a comparable efficiency in microbial identification and quantification. This finding can be easily observed in samples 3, 4, 6 and 7 (A and B), where both techniques detected Lactobacillus with equivalent relative abundances (r=0.9883, p=<0.0001, FIG. 4A and Supplementary 3A). Likewise, both methods yielded similar relative abundances for Gardnerella in samples 1, 2, 3, 5, 6, and 7 (A and B) (r=0.8441, p=0.0169, FIG. 4A). Still, 16S rRNA-seq yielded a lower relative abundance than CiRNAseq for the genera Atopobium, Dialister, Megasphaera, Parvimonas, Prevotella, and Sneathia (FIG. 4A).

Both techniques profiled Gardnerella, Aerococcus, Dialister, Lactobacillus, Megasphaera, Parvimonas, Prevotella, and Sneathia. CiRNAseq also detected Anaerococcus, Atopobium, Fenollaria, and Fusobacterium in higher relative abundances than 16S rRNA-seq (FIG. 4A). Genera Actinomyces, Clostridium, Corynebacterium, Peptoniphilus, and Ureaplasma were detected by 16S rRNA-seq (relative abundances between 0.16% to 1.44%), but not by CiRNAseq (FIG. 4A). From the 30 genera that 16S rRNA-seq yielded ≤0.06% in relative abundances, CiRNAseq was concordant in 26 (87%). In general, 16S rRNA-seq and CiRNAseq were concordant in 34 out of the 43 genera analyzed (79%), illustrating the technique's specificity and sensitivity at the genus level.

TABLE IV
Species-level identification using circular probe-based RNA sequencing.

| Bacteria species | SN1B | SN2B | SN3B | SN4B | SN5B | SN6B | SN7B |
|---|---|---|---|---|---|---|---|
| Aerococcus christensenii | | ● | | | | | |
| Anaerococcus hydrogenalis | | ● | | | | | |
| Anaerococcus tetradius | | ● | | | | | |
| Atopobium vaginae | ● | ● | | | ● | | |
| Dialister micraerophilus | ● | ● | | | ● | | |
| Fenollaria massiliensis | | ● | | | | | |
| Fusobacterium nucleatum | | ● | | | | | |
| Gardnerella vaginalis | ● | ● | ● | | ● | ● | ● |
| Lactobacillus acidophilus | | | ● | | | | |
| Lactobacillus crispatus | | | | ● | | | ● |
| Lactobacillus gasseri | | | | | | | ● |
| Lactobacillus iners | | ● | ● | ● | | ● | |
| Lactobacillus jensenii | | | ● | ● | | | |
| Lactobacillus johnsonii | | | | | | | ● |
| Lactobacillus vaginalis | | | | | | | ● |
| Megasphaera genomosp | ● | ● | | | ● | | |
| type 1 | | | | | | | |
| Parvimonas micra | | ● | | | ● | | |
| Prevotella amnii | | ● | | | ● | | |
| Prevotella bivia | ● | | | | | | |
| Prevotella corporis | | ● | | | | | |
| Prevotella disiens | | ● | | | | | |
| Prevotella timonensis | | ● | | | ● | | |
| Sneathia amnii | | | | | ● | | |
| Sneathia sanguinegens | | | | | ● | | |

To further investigate the species resolution of CiRNAseq in the CVM, we also analyzed samples SN1B to SN7B at this taxonomy level, as shown in FIG. 4B and Table 1. In total, we observed 24 different species. We were able to detect two species of Anaerococcus, seven species of Lactobacillus, five species of Prevotella, and two species of Sneathia (FIG. 4B, Table 1). Therefore, these CiRNAseq results suggest the ability to identifying bacteria at the species level with high specificity in the complex CVM niche.

### CiRNAseq: CVM Changes in Women with hrHPV-Induced Lesions

Several studies suggest that accurate detection of microbial species in the CVM may be relevant for predicting the progression of hrHPV-induced precancerous cervical lesions and cancer [15, 59-61]. To investigate this, we applied CiRNAseq to RNA isolated from cervical smears of hrHPV negative women (considered healthy, n=46) and women with hrHPV positive high-grade cervical intraepithelial neoplasia (CIN2+, n=46).

Unsupervised clustering analysis using URC from each microbial species in individual samples of our cohort is shown to generate three clusters (FIG. 5A). The clusters represented the well-known community state types (CST) [5]. Cluster 1 consisted of 18 samples, of which 72.2% were hrHPV negative, and was characterized by a CST I that is dominated by L. crispatus. Additional Lactobacillus species such as L. iners, L. jensenii, L. ultunensis, and L. acidophilus were also common (FIG. 5A). With a Fisher's exact test, CST I showed a small association to hrHPV negative women (p=0.0639) when compared to hrHPV positive women in cluster 1.

Cluster 2 consisted of 27 samples, of which 20 (74%) were from women with hrHPV-induced high-grade lesions. These women had a CVM consistent with CST IV, characterized by depletion of Lactobacillus species and colonization of mainly anaerobic bacteria such as M. genomosp type 1, G. vaginalis, S. amnii, S. sanguinegens, D. micraerophilus, and A. vaginae. With a Fisher's exact test, CST IV exhibited a significant association to hrHPV positive women (p=0.0055) when compared to hrHPV negative women in cluster 2. The third cluster (3) contained 47 samples, of which 26 (55.3%) were hrHPV negative, and 21 (44.7%) had hrHPV-induced lesions. Women's CVM in cluster 3 were still dominant for Lactobacillus species, and their microbial composition was consistent with other CST such as II (dominance for L. gasseri), III (dominance for L. iners), and V (dominance for L. jensenii) (FIG. 5A).

We also tested our cohort of 92 samples through a Principal Component Analysis (PCA). We determined PC1 and PC2, representing 32.7% and 12.6% of our cohort, respectively (FIG. 5B). Here, we observed a minor separation of samples corresponding to both hrHPV negative and hrHPV positive women with some overlap. After analyzing the loading score of PC1 (Additional File 4), we found that anaerobic bacteria such as M. genomosp type 1 and G. vaginalis showed the higher correlation with PC1, suggesting an association of particular bacterial species with hrHPV status (FIG. 5B).

Although we observed a particular shift in the CVM of samples within clusters 1 and 2, the microbiome composition was ambiguous in cluster 3, possibly due to the presence of different CST in this cluster (FIG. 5A). To further evaluate the microbial composition of our cohort, we performed a supervised average analysis comparing the CVM of hrHPV negative (n=46) and hrHPV positive (n=46) women (Supplementary FIG. 4). This analysis showed that hrHPV negative women were typically colonized with L. acidophilus, L. crispatus, L. jensenii, L. psittaci, L. ultunensis, and L. vaginalis. In contrast, hrHPV positive with high-grade lesions women possessed a more diverse microbiome with anaerobic bacteria such as A. vaginae, D. micraerophilus, G. vaginalis, S. amnii, and S. sanguinegens. Interestingly, L. iners was also present in hrHPV positive women. Other bacteria found in hrHPV positive women included Prevotella species such as P. amnii, P. buccalis, and P. timonensis (Supplementary FIG. 4). To confirm these observations, we performed a Linear discriminant analysis (LDA) effect size (LEfSe) [55] modeling comparing microbiome composition and relative abundance between hrHPV negative (n=45, an outlier was excluded from this analysis) and hrHPV positive samples (n=46) (FIG. 5C). In the hrHPV positive group, this analysis showed higher levels for G. vaginalis, M. genomosp type 1, S. amnii, S. sanguinegens, P. anaerobius, D. micraerophilus, A. vaginae, P. amnii, and P. buccalis (p<0.05) (FIG. 5C and Supplementary 5A-5I). In contrast, in the hrHPV negative group this analysis determined an over-representation of L. acidophilus (p<0.05) (FIG. 5C and Supplementary 5J). Thus, the CVM shift due to hrHPV infection is characterized by the change from a healthy Lactobacillus microbiota to an anaerobic-diverse microbiota that can be explored using CiRNAseq.

### CiRNAseq Profiling Reveals Alterations in the CVM

To further show the significance of CiRNAseq in studying CVM alterations, we examined the two clusters enriched for CST I (1) and CST IV (2) from the analysis described in FIG. 5A. We also assessed the difference in microbial richness, diversity, and relative abundance for L. iners in our cohort's two main groups: hrHPV negative women versus hrHPV positive women with CIN2+.

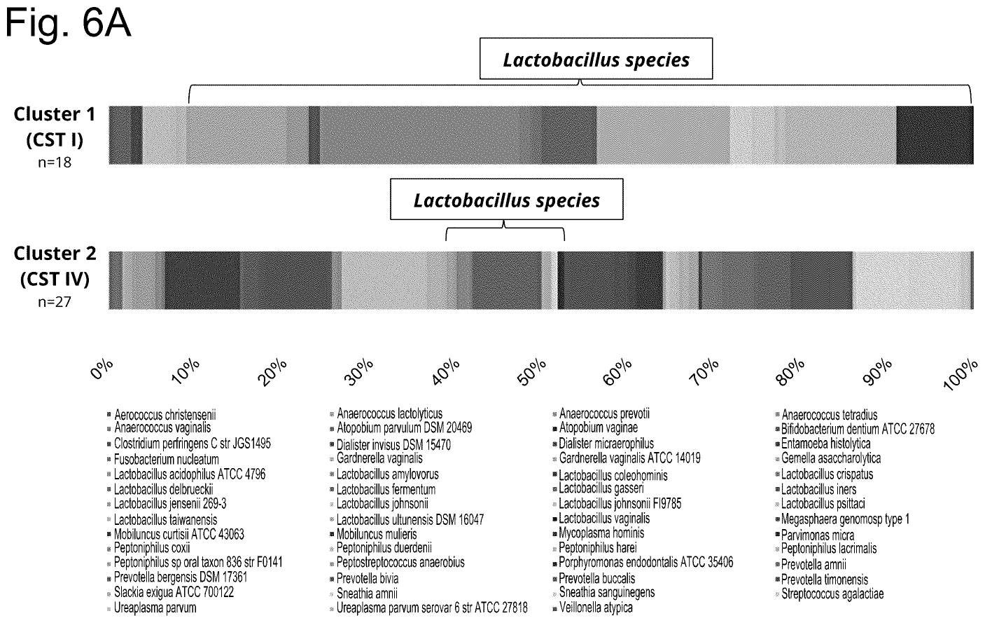

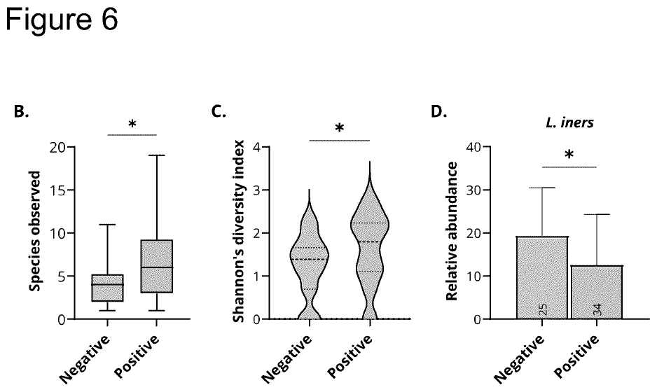

The clusters enriched for CST I and IV had 18 and 27 samples, respectively. The CVM from these two clusters seemingly varied in microbial diversity (FIG. 6A). CST I, containing mostly hrHPV negative women, had a shallow microbial diversity characterized by Lactobacillus species like L. acidophilus, L. crispatus, L. iners, L. jensenii, and L. ultunensis. Therefore, CST I was diverse at the species level but less diverse at the genus level (FIG. 6A). In contrast, within CST IV, consisting of mainly hrHPV positive women, such Lactobacillus species were depleted, and only L. iners continued to be present (FIG. 6A), as described in previous analyses (Figures and Supplementary 4). Moreover, CST IV had a highly diverse microbiome characterized by A. vaginae, D. micraerophilus, G. vaginalis, L. iners, M. genomosp type 1, P. timonensis, S. amnii, S. sanguinegens, and other bacteria as detailed in FIG. 6A. To quantify this observation, we calculated species richness and alpha-diversity, which confirmed that hrHPV negative women had a less rich (mean of 4.2 microbes) and diverse (mean of 1.22) microbiome when compared to hrHPV positive women, mean of 6.6 for richness and 1.60 for alpha-diversity (p=<0.05) (FIGS. 6B and 6C). In conclusion, CiRNAseq let us determine that, besides a CVM shift upon hrHPV infections, there is an alteration of the microbial diversity.

Given that L. iners colonize both hrHPV negative and positive women [59, 62] but did not show a strong association to hrHPV status in our LefSe analysis, we assessed the bacterium abundance independently. To this purpose, we examined our cohort of 92 cervical samples and selected samples for which CiRNAseq identified L. iners. Accordingly, we included 25 hrHPV negative samples, and 34 hrHPV positive samples for this analysis. Following the estimation of relative abundances within the samples, we calculated the mean and significance of the differences, as observed in FIG. 6D. Here, we noticed that L. iners had a higher relative abundance in hrHPV negative women (mean 19.3) when compared to hrHPV positive women (mean 11.9), suggesting that even though it is present in the diverse microbiome of hrHPV positive women, the abundance of this specie decreases upon infection (FIG. 6D).

### Discussion

16S rRNA gene sequencing is the most widely employed method for microbiota analysis and can provide information on the CVM at the genus resolution [23, 27, 43, 63]. In this application, we introduce a novel targeted sequencing method with sufficient resolution and specificity to enable the profiling of cervicovaginal microbiota with similar performance to 16S rRNA-seq, but with additional advantage of very high-throughput profiling. Using CiRNAseq, we show that hrHPV positive women with high-grade cervical intraepithelial neoplasia acquire a characteristic CST IV microbiome as observed by earlier 16S rRNA-seq studies.

CiRNAseq achieves a higher sensitivity than 16S rRNA-seq, which is a result of the underlying smMIP technique in which the same molecule is amplified multiple times. Our findings detailing the identification and quantification of genera such as Lactobacillus and Gardnerella with equivalent results to 16S rRNA-seq corroborate our technique's specificity at the genus level. Nonetheless, since CiRNAseq uses two and more VRs for microbiome profiling and can target both SSU and LSU for identifying some species, its resolution increases to the species taxonomy rank, but further studies on the level of classification confidence at species resolution is warranted. This way, we demonstrated that in fact such genera corresponded to specific species such as L. crispatus, L. iners, L. jensenii, or G. vaginalis, which are extremely relevant for women's cervical health and disease [4, 8, 64, 65]. Thus, our technology confirms recent studies highlighting the advantage of targeting and combining multiple VRs to improve the resolution of microbiome profiling [29, 66].

CiRNAseq showed that the CVM of women shifts from a healthy Lactobacillus-dominated microbiome (CST I) to an anaerobic-diverse microbiome (CST IV) upon severe hrHPV infection. Changes in vaginal pH have been associated with the microbial composition, particularly with depletion of Lactobacillus species and the enhancement of facultative anaerobic bacteria such as G. vaginalis, D. micraerophilus, A. vaginae, Megasphaera spp., and Prevotella spp. (CST IV) [5, 67, 68]. Interestingly, Mitra et al. recently described that CST IV is strongly associated with hrHPV-induced high-grade cervical intraepithelial neoplasia [15]. Since we also observe this association in our cohort of samples, it corroborates and validates the findings obtained with CiRNAseq. On the other hand, the role of individual species in the microbiome shift still remains unclear. Recent studies suggest that G. vaginalis drives the vaginal dysbiosis in hrHPV-infected women and exhibits an immunosuppressive role in the vagina, which could explain the higher abundance of G. vaginalis in hrHPV positive women described in our study [2, 59]. Therefore, identifying individual species within the CVM may elucidate the roles of particular bacteria in the microbiome and provide alternative treatment strategies to prevent disease [69]. Furthermore, understanding the CVM shift at this taxonomic rank may lead to identifying microbiome profiles that could act as predictive biomarkers for women at risk of developing cervical cancer [15, 16, 18, 70, 71]. Additional studies with a larger cohort of samples are needed to clarify whether the species or CST described in the current study possess such function and explain how they would associate with the effect of hrHPV infections.

Using CiRNAseq, we also determined that hrHPV positive women with CIN2+ have a more diverse CVM than hrHPV negative women. In addition, our data suggest that several Lactobacillus species colonize hrHPV negative women, and thus, they are more diverse at the species level than at the genus level, as previously reported [72]. Interestingly, we observed that L. acidophilus was highly abundant in hrHPV negative women considered healthy, which could be attributed to the antimicrobial activities of this Lactobacillus species [73, 74]. Additionally, we found higher levels of L. iners within the CVM of hrHPV positive women. This finding is in line with previous research reporting that L. iners may not be as protective as other Lactobacillus species because particular L. iners strains have been associated with vaginal dysbiosis [3, 14]. Some studies suggest that D-lactate, produced by L. crispatus and not L. iners, enhances the trapping of HIV in the cervicovaginal mucus [11, 75]. By this mechanism, L. crispatus, but not L. iners, could also protect the basal epithelium from infection with hrHPV. Furthermore, the lower abundance of L. iners in smears from women with hrHPV-induced high-grade lesions could also be attributed to changes in the vaginal pH and a decline in the metabolic activities of L. iners [65, 68]. As far as we know, this is the first study to report a higher abundance of L. acidophilus in hrHPV negative women and a lower abundance of L. iners in hrHPV positive women with high-grade cervical intraepithelial neoplasia. Further studies are needed to investigate how the relative abundances of both L. acidophilus and L. iners species associate with hrHPV-induced malignancy [76].

The strengths of this study and CiRNAseq technique are the successful validation tests with microbiota mocks and cervical samples.

In summary, CiRNAseq is a highly promising technology with the resolution and specificity for high-throughput sequencing, which makes it a remarkable tool for uncovering the role of the CVM in health and disease

### REFERENCES

1. Ferlay J, e.a. Global Cancer Observatory: Cancer Today. Lyon, France: International Agency for Research on Cancer. 2018 [cited 2020 November 23]; Available from: https://gco.iarc.fr/today.
2. Murphy, K. and C.M. Mitchell, The Interplay of Host Immunity, Environment and the Risk of Bacterial Vaginosis and Associated Reproductive Health Outcomes. The Journal of Infectious Diseases, 2016. 214(suppl_1): p. S29-S35.
3. Brotman, R.M., et al., Interplay Between the Temporal Dynamics of the Vaginal Microbiota and Human Papillomavirus Detection. The Journal of Infectious Diseases, 2014. 210(11): p. 1723-1733.
4. Moscicki, A.-B., et al., Cervical-Vaginal Microbiome and Associated Cytokine Profiles in a Prospective Study of HPV 16 Acquisition, Persistence, and Clearance. Frontiers in Cellular and Infection Microbiology, 2020. 10(528).
5. Ravel, J., et al., Vaginal microbiome of reproductive-age women. Proceedings of the National Academy of Sciences, 2011. 108(Supplement 1): p. 4680.
6. Chaban, B., et al., Characterization of the vaginal microbiota of healthy Canadian women through the menstrual cycle. Microbiome, 2014. 2(1): p. 23.
7. MacIntyre, D.A., et al., The vaginal microbiome during pregnancy and the postpartum period in a European population. Scientific Reports, 2015. 5(1): p. 8988.
8. Vodstrcil, L.A., et al., The influence of sexual activity on the vaginal microbiota and Gardnerella vaginalis clade diversity in young women. PLOS ONE, 2017. 12(2): p. e0171856.
9. Koedooder, R., et al., The vaginal microbiome as a predictor for outcome of in vitro fertilization with or without intracytoplasmic sperm injection: a prospective study. Human Reproduction, 2019. 34(6): p. 1042-1054.
10. Gajer, P., et al., Temporal Dynamics of the Human Vaginal Microbiota. Science Translational Medicine, 2012. 4(132): p. 132ra52.
11. Nunn, K.L., et al., Enhanced Trapping of HIV-1 by Human Cervicovaginal Mucus Is Associated with &lt;span class=&quot;named-content genus-species&quot; id=&quot;named-content-1&quot;&gt;Lactobacillus crispatus&lt;/span&gt;-Dominant Microbiota. mBio, 2015. 6(5): p. e01084-15.
12. Onderdonk, A.B., M.L. Delaney, and R.N. Fichorova, The Human Microbiome during Bacterial Vaginosis. Clinical Microbiology Reviews, 2016. 29(2): p. 223.
13. Liu, M.-B., et al., Diverse Vaginal Microbiomes in Reproductive-Age Women with Vulvovaginal Candidiasis. PLOS ONE, 2013. 8(11): p. e79812.
14. Mitra, A., et al., The vaginal microbiota, human papillomavirus infection and cervical intraepithelial neoplasia: what do we know and where are we going next? Microbiome, 2016. 4(1): p. 58.
15. Mitra, A., et al., Cervical intraepithelial neoplasia disease progression is associated with increased vaginal microbiome diversity. Scientific Reports, 2015. 5(1): p. 16865.
16. Molina, M.A., et al., Cervical cancer risk profiling: molecular biomarkers predicting the outcome of hrHPV infection. Expert Review of Molecular Diagnostics, 2020: p. 1-22.
17. Kyrgiou, M., A. Mitra, and A.-B. Moscicki, Does the vaginal microbiota play a role in the development of cervical cancer? Translational Research, 2017. 179: p. 168-182.
18. Mitra, A., et al., The vaginal microbiota associates with the regression of untreated cervical intraepithelial neoplasia 2 lesions. Nature Communications, 2020. 11(1): p. 1999.
19. Bik, E.M., et al., A novel sequencing-based vaginal health assay combining self-sampling, HPV detection and genotyping, STI detection, and vaginal microbiome analysis. PLOS ONE, 2019. 14(5): p. e0215945.
20. Yang, Q., et al., The Alterations of Vaginal Microbiome in HPV16 Infection as Identified by Shotgun Metagenomic Sequencing. Frontiers in Cellular and Infection Microbiology, 2020. 10: p. 286.
21. Berman, H.L., M.R. McLaren, and B.J. Callahan, Understanding and interpreting community sequencing measurements of the vaginal microbiome. BJOG: An International Journal of Obstetrics & Gynaecology, 2020. 127(2): p. 139-146.
22. Hong, K.H., et al., Analysis of the Vaginal Microbiome by Next-Generation Sequencing and Evaluation of its Performance as a Clinical Diagnostic Tool in Vaginitis. Annals of Laboratory Medicine, 2016. 36(5): p. 441-449.
23. Clarridge, J.E., Impact of 16S rRNA Gene Sequence Analysis for Identification of Bacteria on Clinical Microbiology and Infectious Diseases. Clinical Microbiology Reviews, 2004. 17(4): p. 840.
24. White, T., et al., Amplification and Direct Sequencing of Fungal Ribosomal RNA Genes for Phylogenetics. 1990. p. 315-322.
25. Caporaso, J.G., et al., Ultra-high-throughput microbial community analysis on the Illumina HiSeq and MiSeq platforms. The ISME Journal, 2012. 6(8): p. 1621-1624.
26. Hugerth, L.W., et al., Assessment of &lt;em&gt;ln Vitro&lt;/em&gt; and &lt;em&gt;ln Silico&lt;/em&gt; Protocols for Sequence-Based Characterization of the Human Vaginal Microbiome. mSphere, 2020. 5(6): p. e00448-20.
27. Graspeuntner, S., et al., Selection of validated hypervariable regions is crucial in 16S-based microbiota studies of the female genital tract. Scientific Reports, 2018. 8(1): p. 9678.
28. Zeeuwen, P.L.J.M., et al., Reply to Meisel et&#xa0;al. Journal of Investigative Dermatology, 2017. 137(4): p. 961-962.
29. Pinna, N.K., et al., Can Targeting Non-Contiguous V-Regions With Paired-End Sequencing Improve 16S rRNA-Based Taxonomic Resolution of Microbiomes?: An In Silico Evaluation. Frontiers in Genetics, 2019. 10: p. 653.
30. de Bitter, T., et al., Profiling of the metabolic transcriptome via single molecule molecular inversion probes. Scientific Reports, 2017. 7(1): p. 11402.
31. van den Heuvel, C.N.A.M., et al., Molecular Profiling of Druggable Targets in Clear Cell Renal Cell Carcinoma Through Targeted RNA Sequencing. Frontiers in Oncology, 2019. 9: p. 117.
32. van den Heuvel, C.N.A.M., et al., Quantification and localization of oncogenic receptor tyrosine kinase variant transcripts using molecular inversion probes. Scientific Reports, 2018. 8(1): p. 7072.
33. Lenting, K., et al., Mapping actionable pathways and mutations in brain tumours using targeted RNA next generation sequencing. Acta Neuropathologica Communications, 2019. 7(1): p. 185.
34. van den Heuvel, C.N.A.M., et al., RNA-based high-risk HPV genotyping and identification of high-risk HPV transcriptional activity in cervical tissues. Modern Pathology, 2020. 33(4): p. 748-757.
35. Boyle, E.A., et al., MIPgen: optimized modeling and design of molecular inversion probes for targeted resequencing. Bioinformatics, 2014. 30(18): p. 2670-2672.
36. Eijkelenboom, A., et al., Reliable Next-Generation Sequencing of Formalin-Fixed, Paraffin-Embedded Tissue Using Single Molecule Tags. The Journal of Molecular Diagnostics, 2016. 18(6): p. 851-863.
37. Martin, D.H. and J.M. Marrazzo, The Vaginal Microbiome: Current Understanding and Future Directions. The Journal of Infectious Diseases, 2016. 214(suppl_1): p. S36-S41.
38. Fettweis, J., et al., The Vaginal Microbiome: Disease, Genetics and the Environment. Nature Precedings, 2010.
39. Coordinators, N.R., Database resources of the National Center for Biotechnology Information. Nucleic acids research, 2016. 44(D1): p. D7-D19.
40. Drost, H.-G. and J. Paszkowski, Biomartr: genomic data retrieval with R. Bioinformatics, 2017. 33(8): p. 1216-1217.
41. Cock, P.J.A., et al., Biopython: freely available Python tools for computational molecular biology and bioinformatics. Bioinformatics, 2009. 25(11): p. 1422-1423.
42. Quinlan, A.R. and I.M. Hall, BEDTools: a flexible suite of utilities for comparing genomic features. Bioinformatics, 2010. 26(6): p. 841-842.
43. Ludwig, W. and K.H. Schleifer, Bacterial phylogeny based on 16S and 23S rRNA sequence analysis. FEMS Microbiology Reviews, 1994. 15(2-3): p. 155-173.
44. Yilmaz, P., et al., The SILVA and “All-species Living Tree Project (LTP)” taxonomic frameworks. Nucleic Acids Research, 2014. 42(D1): p. D643-D648.
45. Morgulis, A., et al., Database indexing for production MegaBLAST searches. Bioinformatics, 2008. 24(16): p. 1757-1764.
46. Li, H. and R. Durbin, Fast and accurate short read alignment with Burrows-Wheeler transform. Bioinformatics, 2009. 25(14): p. 1754-1760.
47. Hamady, M. and R. Knight, Microbial community profiling for human microbiome projects: Tools, techniques, and challenges. Genome Research, 2009. 19(7): p. 1141-1152.
48. Ederveen, T.H.A., et al., A generic workflow for Single Locus Sequence Typing (SLST) design and subspecies characterization of microbiota. Scientific Reports, 2019. 9(1): p. 19834.
49. Zhang, J., et al., PEAR: a fast and accurate Illumina Paired-End reAd mergeR. Bioinformatics, 2014. 30(5): p. 614-620.
50. Caporaso, J.G., et al., QIIME allows analysis of high-throughput community sequencing data. Nature Methods, 2010. 7(5): p. 335-336.
51. Metsalu, T. and J. Vilo, ClustVis: a web tool for visualizing clustering of multivariate data using Principal Component Analysis and heatmap. Nucleic Acids Research, 2015. 43(W1): p. W566-W570.
52. Dhakan, D.B., et al., The unique composition of Indian gut microbiome, gene catalogue, and associated fecal metabolome deciphered using multi-omics approaches. GigaScience, 2019. 8(3).
53. Watanabe, H., et al. Minor taxa in human skin microbiome contribute to the personal identification. PloS one, 2018. 13, e0199947 DOI: 10.1371/journal.pone.0199947.
54. Weiss, S., et al., Normalization and microbial differential abundance strategies depend upon data characteristics. Microbiome, 2017. 5(1): p. 27.
55. Segata, N., et al., Metagenomic biomarker discovery and explanation. Genome Biology, 2011. 12(6): p. R60.
56. Blazewicz, S.J., et al., Evaluating rRNA as an indicator of microbial activity in environmental communities: limitations and uses. The ISME Journal, 2013. 7(11): p. 2061-2068.
57. Ghartey, J.P., et al., Lactobacillus crispatus dominant vaginal microbiome is associated with inhibitory activity of female genital tract secretions against Escherichia coli. PLOS One, 2014. 9(5): p. e96659.
58. Cools, P., The role of Escherichia coli in reproductive health: state of the art. Research in Microbiology, 2017. 168(9): p. 892-901.
59. Usyk, M., et al., Cervicovaginal microbiome and natural history of HPV in a longitudinal study. PLOS Pathogens, 2020. 16(3): p. e1008376.
60. Dareng, E.O., et al., Prevalent high-risk HPV infection and vaginal microbiota in Nigerian women. Epidemiology and infection, 2016. 144(1): p. 123-137.
61. Chen, Y., et al., Association between the vaginal microbiome and high-risk human papillomavirus infection in pregnant Chinese women. BMC Infectious Diseases, 2019. 19(1): p. 677.
62. Kwasniewski, W., et al., Microbiota dysbiosis is associated with HPV-induced cervical carcinogenesis. Oncol Lett, 2018. 16(6): p. 7035-7047.
63. Shin, J., et al., Analysis of the mouse gut microbiome using full-length 16S rRNA amplicon sequencing. Scientific Reports, 2016. 6(1): p. 29681.
64. van der Veer, C., et al., Comparative genomics of human Lactobacillus crispatus isolates reveals genes for glycosylation and glycogen degradation: implications for in vivo dominance of the vaginal microbiota. Microbiome, 2019. 7(1): p. 49.
65. Borgdorff, H., et al., Unique Insights in the Cervicovaginal Lactobacillus iners and L. crispatus Proteomes and Their Associations with Microbiota Dysbiosis. PloS one, 2016. 11(3): p. e0150767-e0150767.
66. Fuks, G., et al., Combining 16S rRNA gene variable regions enables high-resolution microbial community profiling. Microbiome, 2018. 6(1): p. 17.
67. Nelson, T.M., et al., Vaginal biogenic amines: biomarkers of bacterial vaginosis or precursors to vaginal dysbiosis? Frontiers in physiology, 2015. 6: p. 253-253.
68. Clarke, M.A., et al., A large, population-based study of age-related associations between vaginal pH and human papillomavirus infection. BMC Infectious Diseases, 2012. 12(1): p. 33.
69. Lavitola, G., et al., Effects on Vaginal Microbiota Restoration and Cervical Epithelialization in Positive HPV Patients Undergoing Vaginal Treatment with Carboxy-Methyl-Beta-Glucan. BioMed Research International, 2020. 2020: p. 5476389.
70. Chao, X., et al., Research of the potential biomarkers in vaginal microbiome for persistent high-risk human papillomavirus infection. Annals of Translational Medicine, 2020. 8(4): p. 100.
71. Curty, G., et al., Analysis of the cervical microbiome and potential biomarkers from postpartum HIV-positive women displaying cervical intraepithelial lesions. Scientific Reports, 2017. 7(1): p. 17364.
72. Witkin, S.S. and I.M. Linhares, Why do lactobacilli dominate the human vaginal microbiota? BJOG: An International Journal of Obstetrics & Gynaecology, 2017. 124(4): p. 606-611.
73. Chee, W.J.Y., S.Y. Chew, and L.T.L. Than, Vaginal microbiota and the potential of Lactobacillus derivatives in maintaining vaginal health. Microbial Cell Factories, 2020. 19(1): p. 203.
74. Satpute, S.K., et al., Inhibition of pathogenic bacterial biofilms on PDMS based implants by L. acidophilus derived biosurfactant. BMC Microbiology, 2019. 19(1): p. 39.
75. Reimers, L.L., et al., The Cervicovaginal Microbiota and Its Associations With Human Papillomavirus Detection in HIV-Infected and HIV-Uninfected Women. The Journal of Infectious Diseases, 2016. 214(9): p. 1361-1369.
76. Petrova, M.I., et al., <em>Lactobacillus iners</em>: Friend or Foe? Trends in Microbiology, 2017. 25(3): p. 182-191.
77. Hornung, B.V.H., R.D. Zwittink, and E.J. Kuijper, Issues and current standards of controls in microbiome research. FEMS Microbiology Ecology, 2019. 95(5).
78. Johnson, J.S., et al., Evaluation of 16S rRNA gene sequencing for species and strain-level microbiome analysis. Nature Communications, 2019. 10(1): p. 5029.
79. Thomas, A.M. and N. Segata, Multiple levels of the unknown in microbiome research. BMC Biology, 2019. 17(1): p. 48.
80. Ravel, J. and R.M. Brotman, Translating the vaginal microbiome: gaps and challenges. Genome Medicine, 2016. 8(1): p. 35.

1. Method for in vitro profiling of a complex microbiome comprising:
providing a sample from the complex microbiome
performing RNA profiling on the sample by multiplex RNA sequencing, targeting multiple regions of interest, wherein a region of interest preferably is a gene of interest, or a part thereof.

2. The method according to claim 1, wherein the multiplex RNA sequencing is performed using molecular inversion probes (MIPs).

3. The method according to claim 1, wherein the method is for profiling bacterial DNA present in the complex microbiome.

4. The method according to claim 1, wherein the complex microbiome is isolated from the gut, skin, bladder, skin, mouth, nose, ears, lungs or the cervicovaginal area, preferably from the cervicovaginal area.

5. The method according to claim 1, wherein the multiplex RNA sequencing is performed using at least one MIP selected from the group listed in table II.

6. Method according to claim 5, wherein the method comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 MIPs from the from the group listed in table II.

7. The method according to claim 1, wherein the genes of interest are selected from the group consisting of genes encoding enzymes that are involved in tyrosine metabolism, tryptophan metabolism, bile acid metabolism, fatty acid metabolism, amino acid metabolism; 16S rRNA genes; 23S rRNA genes and genes encoding toxins.

8. The method according to claim 1, wherein the genes of interest are selected for their discriminating capacity to identify a microbial genus, a microbial species or a microbial strain.

9. The method according to claim 1, wherein the method is for identifying the relationships between microbial genus, a microbial species or a microbial strain.

10. The method according to claim 9, wherein the method is for identifying microbial compositions and functions in the mouth, airways, gut, cervix, urinary bladder, skin, ears and eyes that are diagnostic and prognostic for disease, including but not limited to tooth decay, head and neck cancer, pneumonia, eczema, lychen sclerosis, bladder cancer, bladder infection, cervical intraepithelial neoplasia, cervical cancer, inflammatory bowel disease, colon adenomas, colon cancer.

11. The method according to claim 1, wherein the method is for the identification of a diet or therapy for treatment of a disease or disorder including but not limited to tooth decay, head and neck cancer, pneumonia, eczema, lychen sclerosis, bladder cancer, bladder infection, cervicovaginal malignancies or disorders such as cervical intraepithelial neoplasia and cervical cancer, inflammatory bowel disease, colon adenomas, colon cancer and neurological diseases such as Alzheimers disease, Multiple Sclerosis and Parkinson disease.

12. At least one molecular inversion probe selected from the group listed in table II.

13. (canceled)

14. (canceled)

15. A method of detecting the presence or absence of a target nucleic acid in a complex microbiome sample, wherein the method comprises:
a) contacting the sample with at least one molecular inversion probe (MIP), wherein said MIP comprises a first hybridization arm comprising a first sequence complementary to a first region in the target nucleic acid of interest, a second hybridization arm comprising a second sequence complementary to second region in the target nucleic acid of interest and a detectable moiety,
b) extending the extension arm with a DNA polymerase and ligating the extended MIP ends that are hybridized to complementary targets to the ligation arm of said MIPs to form circularized MIPs,
c) purifying the circularized MIP;
d) amplifying the purified circularized MIP, preferably by PCR;
e) optionally, purifying the amplified product containing the MIP sequence
f) subjecting the amplified product to next generation sequencing; and
g) detecting the presence or absence of a target nucleic acid in the sample by detecting the presence or absence of the corresponding amplified MIP sequence,
wherein at least one MIP is selected from the group listed in table II.

16. The method according to claim 15, wherein the complex microbiome is from the cervicovaginal area.
